(19) United States
(12) Patent Application Publication
(10) Pub. No.: US 2024/0252101 A1
(43) Pub. Date: Aug. 1, 2024

(54) SKIN CHARACTERIZATION METHODS AND DEVICES

(71) Applicant: SKINFIELD LTD., Ramat Hasharon (IL)

(72) Inventors: Jonathan MAGAL, Ramat Hasharon (IL); Elad MAGAL, Ramat Hasharon (IL)

(21) Appl. No.: 18/560,939

(22) PCT Filed: Jun. 16, 2022
(86) PCT No.: PCT/IB22/55564

§ 371 Date: Nov. 15, 2023

Related U.S. Application Data
(60) Provisional application No. 63/212,827, filed on Jun. 21, 2021.

(51) Int. Cl. A61B 5/00; A61B 5/103

(52) U.S. Cl. CPC A61B 5/443; A61B 5/1032; A61B 5/6833; A61B 2562/12

(57) ABSTRACT

The invention, in some embodiments, relates to methods and devices that, in some embodiments, allow for the characterization of the skin of a human subject. The results of the characterization of the skin by the method or device may be used, for example, to aid in selecting cosmetics and/or skin-care product and/or providing information necessary for making a personalized cosmetic product and/or skin-care product.

### RELATED APPLICATION

The present application gains priority from U.S. provisional patent application 63/212,827 filed 21 Jun. 2021, which is included by reference as if fully set-forth herein.

### FIELD AND BACKGROUND OF THE INVENTION

The invention, in some embodiments, relates to the field of toiletries and cosmetics and, more particularly but not exclusively, to methods and devices that, in some embodiments, allow for the characterization of the skin of a human subject, especially of the face. The results of the characterization of the skin by the method or device may be used, for example, to aid in selecting cosmetics and/or skin-care product and/or providing information necessarily for making a personalized cosmetic product and/or skin-care product.

Different people have different skin characteristics including: skin moisture (dry skin to hydrated skin), oiliness (sebum production: normal, dry, oily and combination skin types), pH level, scaliness/desquamation and sensitivity.

Most cosmetics are formulated for what is considered normal skin. A typical person samples multiple cosmetic formulations until finding one that is compatible with their skin and then continues using that formulations. Some cosmetics are formulated for and marketed as special for unusual skin, for example, formulated for dry skin or formulated for oily skin. A person who believes that their skin is unusual, e.g., after failing to find a suitable formulation for normal skin, may sample such special formulations and may find one that is suitable.

To assist in marketing special cosmetic formulation and to identify a person's skin type and identify a skin type, there exist skin-testing devices for determining skin characteristics.

Courage+Khazaka electronic GmbH (Köln, Germany) developed special electronic and optical equipment for skin characterization to promote cosmetic products or treatments at the point of sale in malls, public areas, shows, aesthetic salons or at the dermatologist's practice.

The company also provides single-use devices for qualitatively characterizing skin including:

Corneofix® CF 20 to characterize hydration level of dryness or damaged skin being a 2 cm×2 cm, 0.1 mm thick transparent backing foil with an adhesive face. The adhesive face is held in place against the skin to collect dead corneocytes from the top layer of skin. With less hydrated and/or damaged skin, more thick and large corneocytes adhere to the adhesive face whole moist skin shows small regular flakes. Accordingly, the device provides a visualization of skin moisture and scaliness. Similar devices include devices from USP Indicator Solutions (Klagenfurt am Wörthersee, Austria) and the D-Squame® discs from device from Clinical & Derm LLC (Dallas, TX, USA).

Sebufix® SF 16 foil to show the level of skin oiliness being a 1.7 cm×1.7 cm, 0.2 mm thick backing foil with a sebum-absorbing face and an adhesive opposite face. The sebum-absorbing face is defined by a hydrophobic microporous film (e.g., of polypropylene) that is opaque where the micropores are filled with air but transparent where the micropores are filled with an oil such as sebum. While keeping the foil stuck to a user's finger through the adhesive face, the sebum-absorbing face is contacted with the skin to absorb oil therefrom, producing visible spots. More and bigger spots indicate greater sebum gland activity (oily skin) while fewer and smaller spots indicate lesser sebum gland activity (not oily skin). Relevant art for this foil includes U.S. Pat. Nos. 4,532,937, 5,119,828, and 5,935,521 all which are included by reference as if fully set-forth herein. Similar devices include devices from USP Indicator Solutions (Klagenfurt am Wörthersee, Austria) and the Sebutape® Skin Indicator from device from Clinical & Derm LLC (Dallas, TX, USA).

Sebu Pad SP 49 is 1.4 cm diameter, 1 cm thick foam pad with an oil-absorbent face that works in the same manner as the Sebufix® SF 16.

In these devices the level of skin oiliness or the level of skin hydration is evaluated qualitatively by comparison to a reference card or by using image-processing software.

It would be useful to have devices that can conveniently assist in characterizing the skin, for example, when used by a consumer at home.

### SUMMARY OF THE INVENTION

Some embodiments of the invention herein relate to methods and devices that, in some embodiments, allow for the characterization of the skin of a human subject, especially of the face. The results of the characterization of the skin by the method or device may be used, for example, to aid in selecting cosmetics and/or a skin-care product and/or providing information necessarily for making a personalized cosmetic product and/or skin-care product and/or for formulating/providing a skin care routine and/or for dermal analysis.

According to an aspect of some embodiments of the teachings herein, there is provided a device suitable for characterizing skin, comprising:
a body having an upper surface and a lower contact face;
the upper surface and the lower contact face both having an area of not less than 0.2 cm² (equivalent to 0.5 cm diameter circle) and not more than 1020 cm² (equivalent to a 18 cm diameter circle);
the body having a thickness of not less than 30 micrometers and not more than 10 mm, wherein at least a part of the contact face comprises an adhesive-bearing portion that surrounds a surface of a sebum indicator which changes appearance subsequent to absorbing sebum from skin.

In some embodiments, the sebum indicator makes up between about 5% and about 50% of a surface area of the contact face.

In some embodiments, the sebum indicator comprises a sebum-absorbing hydrophobic microporous film. In some embodiments, the sebum indicator comprises a single type of the sebum-absorbing hydrophobic microporous film on the contact face. Alternatively, in some embodiments, the sebum indicator comprises at least two different types of the sebum-absorbing hydrophobic microporous film on the contact face, each different type of microporous film having different sebum-absorbing properties.

In some embodiments, the sebum indicator comprises a microporous film assembly comprising a vertical spacer having a front side and a back side, wherein the microporous film of the device is attached to the front side of the vertical spacer; and wherein the microporous film assembly is attached to other components of the device through the back side of the vertical spacer.

In some embodiments, the device comprises an adhesive layer which constitutes the adhesive-bearing portion of the contact face and which also contacts the back side of the vertical spacer so as to maintain the microporous film on the contact face.

In some embodiments, the vertical spacer has a thickness of between about 50 micrometers and about 250 micrometers.

In some embodiments, the adhesive-bearing portion of the contact face includes at least two differently-colored parts when viewed from perpendicular towards the contact face:
a dark-colored part of the contact face constituting a hydration-characterizing portion of the contact face; and
a light-colored part of the contact face constituting a pollution-characterizing portion of the contact face.

According to an aspect of some embodiments of the teachings herein, there is also provided a device suitable for characterizing skin, comprising:
a body having an upper surface and a lower contact face;
the upper surface and the lower contact face both having an area of not less than 0.2 cm² (equivalent to 0.5 cm diameter circle) and not more than 1020 cm² (equivalent to a 18 cm diameter circle);
the body having a thickness of not less than 30 micrometers and not more than 10 mm, wherein at least a part of the contact face comprises a surface of a sebum indicator which changes appearance subsequent to absorbing sebum from skin, the sebum indicator comprising at least two different types of the sebum-absorbing hydrophobic microporous film on the contact face, each different type of microporous film having different sebum-absorbing properties.

According to an aspect of some embodiments of the teachings herein, there is also provided a device suitable for characterizing skin, comprising:
a body having an upper surface and a lower contact face;
the upper surface and the lower contact face both having an area of not less than 0.2 cm² (equivalent to 0.5 cm diameter circle) and not more than 1020 cm² (equivalent to a 18 cm diameter circle);
the body having a thickness of not less than 30 micrometers and not more than 10 mm, wherein at least a part of the contact face comprises an adhesive-bearing portion that includes at least two differently-colored parts when viewed from perpendicular towards the contact face:
a dark-colored part of the contact face constituting a hydration-characterizing portion of the contact face; and
a light-colored part of the contact face constituting a pollution-characterizing portion of the contact face.

In some embodiments, a device according to the teachings herein has at least one of: the upper surface of the body being non-glare; and the contact face being non-glare.

In some embodiments, the upper surface of the body comprises at least one image-correction feature that assists in computerized correction of the color and/or white balance of an acquired image in which the upper surface appears.

In some embodiments, the contact face comprises at least one image-correction feature that assists in computerized correction of a distortion of a portion of an acquired image that corresponds to the contact face.

In some embodiments, the body comprises a supporting sheet which provides the body with at least one property selected from the group consisting of being not-stretchy, flexibility to conform to the irregularly curved surface of human skin, stiffness and elasticity.

According to an aspect of some embodiments of the teachings herein, there is also provided a method for making a device comprising a sebum indicator as described herein, the method comprising:
a. providing a sheet of material suitable for being a supporting sheet of a body of the device having a front side and a back side;
b. providing a microporous film assembly comprising a vertical spacer having a front side and a back side, with a microporous film attached to the front side of the vertical spacer;
c. covering the front side of the sheet of material with a layer of adhesive;
d. contacting the back side of the vertical spacer of the microporous film assembly with the layer of adhesive covering the front side of the sheet of material where the microporous film assembly is surrounded by a surface of the layer of adhesive, thereby attaching the microporous film assembly to the front side of the sheet of material; and
e. cutting the sheet of material to yield an incipient device comprising a sebum indicator, wherein the cutting is such that a microporous film assembly constituting an incipient sebum indicator of a device is surrounded by a surface of the layer of adhesive constituting an adhesive-bearing portion of a device. In some embodiments, the sheet of material has a thickness of between about 50 micrometers and about 450 micrometers. Additionally or alternatively, in some embodiments, the method further comprises prior to ‘c’ applying a colorant to the front side of the sheet of material having a desired color pattern.

According to an aspect of some embodiments of the teachings herein, there is also provided a method for making a microporous film assembly for use as a sebum indicator comprising:
providing a sheet of material suitable for being a vertical spacer having a front side and a back side;
covering the front side of the sheet with a layer of adhesive;
adhering, to the layer of adhesive covering the front side of the sheet, a sheet of sebum-absorbing microporous film to make a laminated workpiece; and
cutting the laminated workpiece to make a microporous film assembly.

In some embodiments, the adhesive layer covering the front side of the sheet has a thickness of between about 15 micrometers and about 30 micrometers. In some embodiments, the sheet of material has a thickness of between about 50 micrometers and about 250 micrometers.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale. In the Figures:

FIGS. 1A, 1B, 1C and 1D schematically depict an embodiment of a device according to the teachings herein having a pollution-characterizing portion and a hydration-characterizing portion in bottom view without a protective cover (FIG. 1A), in bottom view with a protective cover (FIG. 1B), in top view (FIG. 1C), and in schematic side cross section (FIG. 1D);

FIGS. 2A, 2B, 2C and 2D schematically depict an additional embodiment of a device according to the teachings herein having a pollution-characterizing portion and a hydration-characterizing portion in bottom view without a protective cover (FIG. 2A), in bottom view with a protective cover (FIG. 2B), in top view (FIG. 2C), and in schematic side cross section (FIG. 2D);

FIGS. 3A and 3B schematically depict an additional embodiment of a device according to the teachings herein having a sebum-characterizing portion in bottom view without a protective cover (FIG. 3A) and in schematic side cross section (FIG. 3B);

FIGS. 4A and 4B schematically depict an additional embodiment of a device according to the teachings herein having a sebum-characterizing portion in bottom view without a protective cover (FIG. 4A) and in schematic side cross section (FIG. 4B);

FIGS. 5A, 5B and 5C schematically depict an additional embodiment of a device according to the teachings herein having two sebum characterizing portions in bottom view without a protective cover (FIG. 5A), in schematic side cross section (FIG. 5B) and schematic exploded view (FIG. 5C); and

FIGS. 6A, 6B, 6C, 6D and 6E depict the contact face of different devices according to the teachings herein.

### DESCRIPTION OF SOME EMBODIMENTS OF THE INVENTION

Some embodiments of the invention herein relate to methods and devices that, in some embodiments, allow for the characterization of the skin of a human subject, especially of the face. The results of the characterization of the skin by the method or device may be used, for example, to aid in selecting cosmetics and/or skin-care product and/or providing information necessarily for making a personalized cosmetic product and/or a skin-care product and/or for formulating/providing a skin care routine and/or for dermal analysis. In some embodiments, the device can be used by an untrained person, for example, at home by a consumer.

The principles, uses and implementations of the teachings of the invention may be better understood with reference to the accompanying description and figures. Upon perusal of the description and figures present herein, one skilled in the art is able to implement the teachings of the invention without undue effort or experimentation. In the figures, like reference numerals refer to like parts throughout.

Before explaining at least one embodiment of the invention in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

The teachings herein disclose a number of exemplary embodiments of a device that is useful for characterizing the skin.

### Two-Color Background Adhesive Device

In some embodiments, the teachings herein provide for a device having a thin body that has an upper surface and a lower surface (hereinfurther called the contact face). At least part of the contact face, called the adhesive-bearing portion, is covered with adhesive. When viewed from perpendicular to the contact face, the adhesive-bearing portion includes at least two different-colored parts: a dark-colored part constituting a hydration-characterizing portion of the contact face; and a light-colored part constituting a pollution-characterizing portion of the contact face.

For use, the contact face is pressed against a human skin surface (e.g., the skin of a forehead or cheek). The adhesive-bearing portion of the contact face maintains the contact face attached to the human skin for a period of time, typically for at least 5 seconds. During this time, particles of dirt and pollution as well as dead skin cells such as corneocytes stick to the adhesive of the adhesive-bearing portion of the contact face.

When desired, the user removes the device from the skin (preferably by peeling off, as known in the art of adhesive bandages). The contact face is then inspected in any suitable way, in preferred embodiments by capturing a digital image of the contact face followed by computerized analysis of the captured digital image.

Dirt and pollution particles are typically dark-colored so are clearly observable against the light-colored pollution-characterizing portion of the contact face. The greater the number of dark-colored particles detected against the light-background of the pollution-characterizing portion, the more polluted the skin is.

Dead skin cells such as corneocytes are typically light-colored so are clearly observable against the dark-colored hydration-characterizing portion of the contact face. If a relatively large number of light-colored particles are detected against the dark-background of the hydration-characterizing portion, it is understood that the skin is dry and scaly, easily shedding dead skin cells. In contrast, if relatively few light-colored particles are detected against the dark background of the hydration-characterizing portion, it is understood that the skin is hydrated and does not easily shed skin cells.

Accordingly, inspection of the contact face comprising the two differently-colored parts, especially computerized analysis of a digital image of the contact face, allows characterization of the level of pollution and the level of hydration of the skin as known in the art. The computerized analysis can be configured to be quantitative, qualitative, or both.

Thus, according to an aspect of some embodiments of the teachings herein, there is provided a device suitable for characterizing skin, comprising:
a body having an upper surface and a lower contact face;
the upper surface and the lower contact face both having an area of not less than 0.2 cm² (equivalent to 0.5 cm diameter circle) and not more than 1020 cm² (equivalent to a 18 cm diameter circle);
the body having a thickness of not less than 30 micrometers and not more than 10 mm, wherein at least a part of the contact face comprises an adhesive-bearing portion that includes at least two differently-colored parts when viewed from perpendicular towards the contact face:
a dark-colored part of the contact face constituting a hydration-characterizing portion of the contact face; and
a light-colored part of the contact face constituting a pollution-characterizing portion of the contact face.

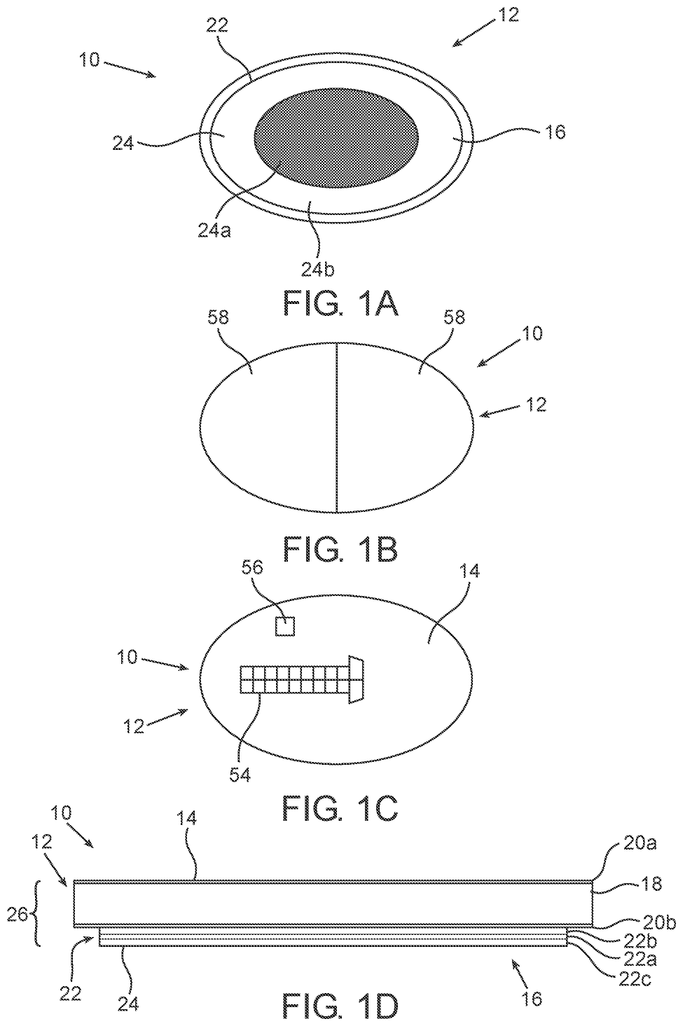

An exemplary embodiment of the device according to the teachings herein, device 10, is schematically depicted in FIG. 1A (bottom view without a protective cover), FIG. 1B (bottom view with a protective cover), FIG. 1C (top view) and FIG. 1D (schematic side cross section). In FIG. 1D, the relative vertical sizes and the vertical scale of the components is distorted so that details of the layers making up the body of device can be seen. Device 10 includes a body 12, an upper surface 14 and a lower contact face 16.

Body 12 comprises a 250 micrometer thick polycarbonate (Lexan®) supporting sheet 18, oval-shaped with dimensions 5 cm×3 cm having a surface area of 11.8 cm².

The top side of supporting sheet 18 is entirely coated with a 20 micrometer thick layer of non-glare ink 20a.

The bottom side of supporting sheet 18 is entirely coated with a 20 micrometer thick layer of non-glare ink 20b.

The ink of ink layers 20a and 20b is applied to the two sides of supporting sheet 18 using any suitable method known in the art of polycarbonate printing, e.g., screen printing or digital printing.

As seen in FIG. 1A, ink layer 20b on the bottom side of supporting sheet 18 includes a central oval of black ink surrounded by an oval ring of white ink.

Attached to the surface of ink layer 20b on the bottom side of supporting sheet 18 is an 4.5 cm×2.5 cm (8.8 cm² surface area) oval of 160 micrometer thick double-sided adhesive film 22 (MED 6000 by Avery-Dennison, Glendale, California, USA) which comprises a 75 micrometer thick transparent polyethylene carrier film 22a coated with a 50 micrometer thick layer 22b and a 35 micrometer thick layer 22c of transparent acrylic adhesive designed for medical use. Adhesive layer 22b secures adhesive film 22 to ink layer 20b.

Adhesive layer 22c defines an adhesive-bearing portion 24 of contact face 16. Since adhesive film 22 is transparent, ink layer 20b is clearly seen through contact face 16. When viewed from perpendicular towards contact face 16 as in FIG. 1A, a central oval part of adhesive-bearing portion 24 is dark-colored (black) and thereby constitutes a hydration-characterizing portion 24a of contact face 16 which is surrounded by an oval ring which is light-colored (white) and thereby constitutes a pollution-characterizing portion 24b of contact face 16.

A thickness 26 of body 12 of device 10 is 450 micrometers between upper surface 14 and the centrally-located adhesive-bearing portion 24 (which makes up 75% of the surface area of contact face 16) and only 290 micrometers at the periphery (which makes up 25% of the surface area of contact face 16).

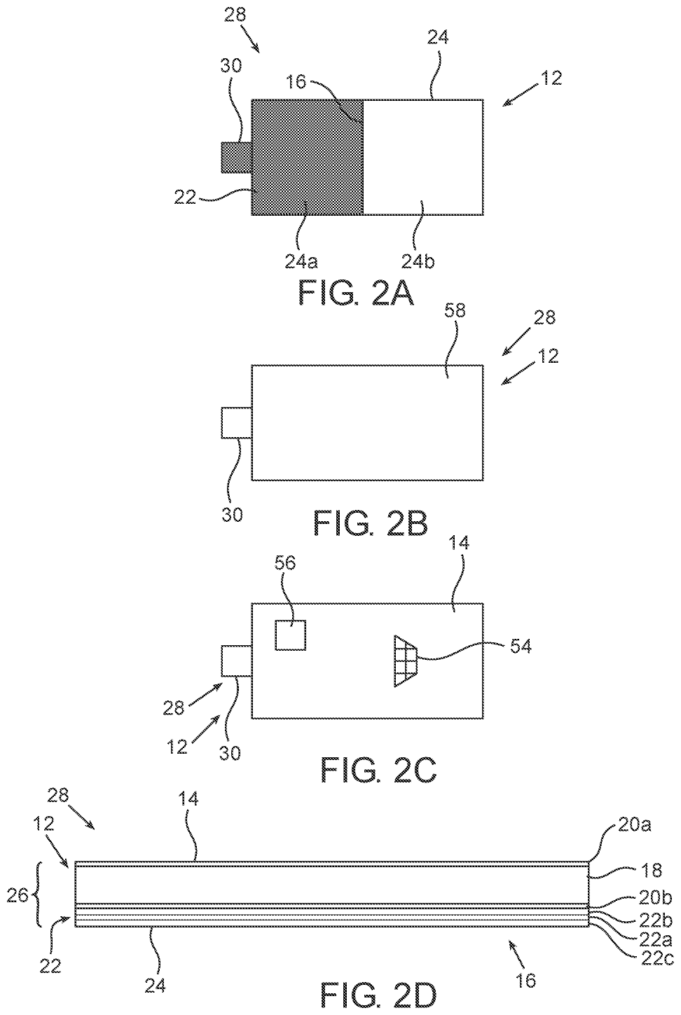

An additional exemplary embodiment of the device according to the teachings herein, device 28, is schematically depicted in FIG. 2A (bottom view without a protective cover), FIG. 2B (bottom view with a protective cover), FIG. 2C (top view) and FIG. 2D (schematic side cross section). In FIG. 2D, the relative vertical sizes and the vertical scale of the components is distorted so that details of the layers making up the body of device can be seen. Device 28 includes a body 12, an upper surface 14 and a lower contact face 16. Body 12 comprises a 250 micrometer thick polycarbonate (Lexan®) supporting sheet 18, rectangle-shaped with dimensions 4 cm×2 cm having a surface area of 8 cm², with a 0.5 cm×0.5 cm tab 30 extending from one of the narrow sides.

As with device 10, in device 28 the top side of supporting sheet 18 is entirely coated with a 20 micrometer thick layer of non-glare ink 20a and the bottom side of supporting sheet 18 is entirely coated with a 20 micrometer thick layer of non-glare ink 20b. As seen in FIG. 2A, ink layer 20b on the bottom side of supporting sheet 18 includes a 2 cm×2 cm square of black ink (with a 0.5 cm×0.5 cm “appendix” at tab 30) and a 2 cm×2 cm square of white ink.

Attached to the surface of ink layer 20b on the bottom side of supporting sheet 18 is an 4 cm×2 cm rectangle of double-sided adhesive film 22 that entirely covers the bottom side except at tab 30. As in device 10, adhesive film 22 is 160 micrometer thick MED 6000 by Avery-Dennison with adhesive layers 22b and 22c. Adhesive layer 22b secures adhesive film 22 to ink layer 20b.

Adhesive layer 22c defines an adhesive-bearing portion 24 of contact face 16. Since adhesive film 22 is transparent, ink layer 20b is clearly seen through contact face 16. When viewed from perpendicular towards contact face 16 as in FIG. 2A, one square part of adhesive-bearing portion 24 is dark-colored (black) and thereby constitutes a hydration-characterizing portion 24a of contact face 16 and another square part of adhesive-bearing portion 24 is light-colored (white) and thereby constitutes a pollution-characterizing portion 24b of contact face 16.

A thickness 26 of device 28 is 450 micrometers between upper surface 14 and centrally-located adhesive-bearing portion 24 (which makes up 94% of the surface area of contact face 16) except for tab 30 (which makes up 6% of the surface area of contact face 16) being only 290 micrometers thick.

### Body

The size of the body is any suitable size. The body is preferably sufficiently large so that a person can easily manipulate the device, including removing from a package, applying to the skin and removing from the skin. The body is preferably not so large as to make it difficult to apply the contact face to the skin. In some preferred embodiments, the size of the body is such that the contact face has a surface area of not less than 0.2 cm² (equivalent to 0.5 cm diameter circle) and not more than 1020 cm² (equivalent to a 18 cm diameter circle). More preferably, the contact face has a surface area of not less than 0.8 cm² (equivalent to 1 cm diameter circle). A contact face 16 of devices 10 (FIG. 1) and 28 (FIG. 2) has a surface area of 11.8 cm² and 8 cm², respectively.

The body is of any suitable thickness. Preferably the body is as thin as possible to be light and so as not to cast a substantial shadow when photographed while adhering to the skin and to make packaging/production simple, yet is preferably sufficiently thick to be easy to manipulate by an average human and to be sufficiently robust and not flimsy so as to tear during ordinary use. In typical embodiments, the body has a thickness of not less than 30 micrometers and not more than 10 mm thick, which is at least partially determined by the material from which the body is made. To make manual handling of the device easier, in some preferred embodiments, the body is not less than about 50 micrometers thick and even not less than about 100 micrometers thick. Additionally or alternatively, in some embodiments the device is not more than about 5 mm thick. Devices 10 (FIG. 1) and 28 (FIG. 2) have a thickness of 450 micrometers (the thickness is labelled 26 in the Figures) which is sufficiently robust and not flimsy, inter alia, due to the rigidity of supporting sheet 18.

Preferably the body is not stretchy, that is to say, a manual stretching force applied by a human in a direction perpendicular to the contact face/top surface leads to stretching of the body by no more than 5%, preferably not more than 3% and even not more than 1%. A stretchy body adheres to a different sized surface of skin depending on how it is applied and distorts when peeled away from the skin, leading to inaccuracies when evaluating a level of skin pollution/skin hydration. Body 12 of devices 10 and 28 is not stretchy due to polycarbonate supporting sheet 18.

The body is preferably sufficiently flexible to conform to the typically irregularly-curved surface of human skin to allow the adhesive-bearing portion of the contact surface to make adequate contact with the skin. Body 12 of devices 10 and 28 is sufficiently flexible due to polycarbonate supporting sheet 18 not being too thick.

In some preferred embodiments, the body is stiff and self-supporting, that is to say, that when the device is held perpendicular to the gravity vector by one end, the body sags by no more than 10% and more preferably by no more than 5% as a result of gravity. Preferably, the body is sufficiently stiff so that when the device adheres to a skin surface and an external force is applied to the outer surface, for example by pressing the device against the skin using a finger, the applied force is distributed over the skin surface and does not remain localized to underneath where the force is applied. In some alternate embodiments, the body is compliant (e.g., like a tissue or prior-art adhesive bandages) allowing the body and contact face to conform to a skin surface and, when the device is laid contact fac-up on a flat surface such as a table, the body of the device adopts the flat shape of the flat surface on which it lays.

In some preferred embodiments, the body is elastic and preferably ordinarily flat, that is to say, when unconstrained by an external force reverts from a curved shape to a flat shape. Such elasticity allows for the acquisition of a clear image of the contact face without fear of shadows or distortions caused by curvature of the body. In such embodiments, it is preferred that the inherent clastic force of the body that causes a bent body to revert to be flat is not so strong as to overcome the adhesion to the skin of the adhesive-bearing portion which would cause the device to fall off of a skin surface prematurely. In some embodiments, the inherent force that causes a bent body to revert to be flat is quantified as the device being configured so that the lower contact face conforms to the curvature of dry skin of the forehead of a human adult when attached to the forehead through the adhesive-bearing portion and the force is sufficiently weak to allow the adhesive-bearing portion to maintain the contact face secured to the skin for at least 5 seconds and even at least 10 seconds, preferably longer. Body 12 of devices 10 (FIG. 1) and 28 (FIG. 2) is clastic and ordinarily flat due to polycarbonate supporting sheet 18. Devices 10 and 28 are configured to allow essentially unlimited duration of adhesion to a curved dry skin surface of an adult human forehead, inter alia, due to the surface area of adhesive-bearing portion 24, the adhesive strength of adhesive layer 22c to dry human skin and the thickness of polycarbonate supporting sheet 18.

Typically, the body is laminated, that is to say, made of one or more layers of a flat material, e.g., sheets, foils or tapes. In some embodiments, the body is not light-transparent. Typically, the body comprises two or more layers of material, e.g., different overlapping sheets, such as devices 10 and 28. In some embodiments, the body has a uniform thickness. Alternatively, in some embodiments, the body has regions with different thicknesses. The bodies of devices 10 and 28 have regions of different thickness, in device 10 the periphery and in device 28, tab 30.

The material from which the body is made is any material or any suitable combination of materials that can fulfill one, some and preferably all of the above-listed requirements such as a plastic (PET, polymethyl acrylate, polyethyl acrylate, polycarbonate, polypropylene), metal and/or metallized plastic.

In some embodiments, the body comprises a supporting sheet (e.g., sheet 18 of device 10 in FIG. 1 and device 28 in FIG. 2) that provides the body with some or all of the desired properties such as being not-stretchy, flexible to conform to the irregularly curved surface of human skin, stiffness and elasticity and resistance to tearing. In preferred embodiments, the supporting sheet is not less than about 50 micrometers thick and not more than about 450 micrometers thick. In some such embodiments, the supporting sheet is not less than about 80 micrometers thick, not less than about 100 micrometers thick, not less than about 150 micrometers thick and even not less than about 200 micrometers thick. Additionally or alternatively, in some such embodiments, the supporting sheet is not more than about 400 micrometers thick and even not more than about 350 micrometers thick.

In preferred embodiments, the supporting sheet is a polycarbonate such as Lexan®. It has been surprisingly found that a polycarbonate such as Lexan® is an exceptionally suitable material from which to make a supporting sheet of a body of a device according to the teachings herein: a supporting sheet of such polycarbonate having the required thickness has been found to help make the body of the device be non-stretchy, flexible, sufficiently conformable, elastic and resistant to tearing. Polycarbonate is available in suitable colors and/or can bear an applied color, and can bear a suitable adhesive.

### Adhesive-Bearing Portion

As noted above, in some embodiments at least part of the contact face of a device of the teachings herein comprises an adhesive-bearing portion that includes at least two different-colored parts when viewed from perpendicular towards the contact face:
a dark-colored part constituting a hydration-characterizing portion of the contact face; and
a light-colored part constituting a pollution-characterizing portion of the contact face.

In some embodiments, the adhesive-bearing portion is the entire contact face. Alternatively, in some embodiments, the adhesive-bearing portion is at least 50% of the contact face, in some such embodiments with at least 5% of the contact face being devoid of adhesive. In preferred embodiments, at least some of the portion of the contact face that is devoid of adhesive is located at the periphery of the contact face to assist in removing the device from skin to which it is adhered, e.g., is part of the periphery of the contact face (e.g., device 10) or is a tab located at the periphery of the contact face (e.g., device 28). In some embodiments, the adhesive-bearing portion is at least 60% and even at least 70% of the contact face.

In device 10 (FIG. 1), 75% of the surface area of contact face 16 is centrally-located adhesive-bearing portion 24 with the remaining peripheral portion 20 of contact face 16 not bearing adhesive. Device 10 is easily removed from a skin surface to which adhered by gripping any part of peripheral portion that is devoid of adhesive and then peeling device 10 away from the skin.

In device 28 (FIG. 2), 94% of the surface area of contact face 16 is adhesive-bearing portion 24 with only tab 30 being 6% of the surface area of contact face 16, devoid of adhesive. Device 28 is easily removed from a skin surface to which adhered by gripping tab 30 and then peeling device 28 away from the skin. Supporting sheet 18 ensures that device 28 is peeled away rather than tears.

In preferred embodiments the adhesive-bearing portion, and preferably the entirety of the contact face of a device, is non-glare (e.g., matte) so when photographed with a flash, the contact face does not specularly reflect light from the flash which reduces the quality of an acquired image. In both device 10 and device 28, the entirety of contact face 16 is non-glare inter alia, due to the application of a layer 20b of non-glare ink to coat contact face 16. A non-glare contact face can be provided in any suitable way, for example, by the appropriate selection of the materials from which the body, any colorants and any adhesive apparent on the contact face. A person having ordinary skill in the art is able to select the appropriate materials from to achieve a non-glare contact face.

### Adhesive

The adhesive present on the adhesive-bearing portion of a contact face of a device according to the teachings herein is any suitable adhesive, preferably medical-grade adhesive, preferably being hypoallergenic. As discussed above, the adhesive is sufficient to keep the device attached to the skin for a suitable period of time, but also allows removing the device from the skin when desired, for example by peeling without causing substantial damage or discomfort, preferably causing no damage or discomfort.

Typically, the device components and construction are such that the device is configured to reversibly adhere to dry human skin for at least 5 and even at least 10 seconds subsequent to application to the skin (although a user can choose to remove the device sooner), which adhering is sufficient to maintain the contact face in intimate contact with the human skin. In this context, “dry human skin” refers to a skin surface without water thereupon, which, if required, can be achieved by blotting the skin surface with a towel, as known in the art of adhesive bandages. The adhesive is configured to reversibly adhere to human skin, allowing a person to manually separate the contact face from the skin without substantial pain or discomfort, as is known in the art of adhesive bandages. Any suitable adhesive may be used, for example, adhesives known in the art of adhesive bandages (e.g., BAND-AID® by Johnson & Johnson, New Brunswick, New Jersey, USA) such as rubber adhesives and acrylates, including methacrylates and epoxy diacrylates. The adhesive is attached to other components of the device in any suitable manner as known in the art of adhesives. It has been found that a particularly suitable manner to attach adhesive to other device components to make an adhesive-bearing surface of a device is by the use of double-sided adhesive film. Suitable such adhesive films are commercially available, inter alia, from Avery-Dennison. In some embodiments, the strength of the adhesive as measured using the loop tack testing method (Finat Test Method No. 9) is at least about 19 N/25 mm (e.g., like the MED 6000 double-coated conformable polyethylene film with acrylic adhesive by Avery Dennison, Glendale, CA, USA) and not more than about 72 N/25 mm (e.g., like the MED 6323 double-coated conformable polyester film with rubber adhesive by Avery Dennison).

### Light-Colored Pollution-Characterizing Portion of the Contact Face

A light-colored part of the adhesive-bearing portion constitutes a pollution-characterizing portion of the contact face.

In some embodiments, the light-colored part of the adhesive-bearing portion is of any color having a lightness of 7-9 in the Munsell color system. In preferred embodiments, the light-colored part of the adhesive-bearing portion is white.

In some embodiments, the adhesive of the light-colored part of the adhesive-bearing portion is light colored.

Additionally or alternatively, in some embodiments, the color of a component underlying the adhesive of the light-colored part of the adhesive-bearing portion is light colored. The color of ink layer 20b underlying transparent double-sided adhesive film 22 that corresponds to light-colored pollution-characterizing portion 24b of devices 10 and 28 is white.

When the light-colored pollution-characterizing portion of a device contacts skin, particles of pollution such as dirt, dust and soot adhere to the adhesive. For characterizing a skin surface for level of pollution, an image of the contact face including the light-colored pollution-characterizing portion is acquired and analyzed by a computer using known image-analysis techniques to give a qualitative and/or quantitative evaluation of the amount of pollution and the cleanliness of the skin because many pollution particles are apparent while skin particles are typically invisible against the underlying light-colored background.

### Dark-Colored Hydration-Characterizing Portion of the Contact Face

A dark-colored part of the adhesive-bearing portion constitutes a hydration-characterizing portion of the contact face.

In some embodiments, the dark-colored part of the adhesive-bearing portion is of any color having a lightness of 0-2 in the Munsell color system. In preferred embodiments, the dark-colored part of the adhesive-bearing portion is black.

In some embodiments, the adhesive of the dark-colored part of the adhesive-bearing portion is dark colored.

Additionally or alternatively, in some embodiments, the color of a component underlying the adhesive of the dark-colored part of the adhesive-bearing portion is dark colored. The color of ink layer 20b underlying transparent double-sided adhesive film 22 that corresponds to dark-colored hydration-characterizing portion 24a of devices 10 and 28 is black.

The hydration-characterizing portion is used to characterize skin scaliness/moisture which are related to skin hydration. Upon contact with skin, loose skin cells such as corneocytes adhere to the adhesive. In normal skin, the adhering skin cells are relatively small and thin. In dry and/or damaged skin, the adhering skin cells are relatively large and thick. For characterizing a skin surface for hydration, an image of the contact face including the dark-colored hydration-characterizing portion is acquired and analyzed by a computer using known image-analysis techniques to give a qualitative and/or quantitative evaluation of the amount and size of the adhered skin cells because the skin cells are apparent against the underlying dark-colored background. The characterization is similar to the characterization of skin hydration performed using the USP Indicator Solutions device.

### Relative Arrangement of the Dark-Colored/Light-Colored Portions

The light-colored pollution-characterizing portion and the dark-colored hydration-characterizing portion of the contact face are of any suitable shape and arranged on a contact face in any suitable pattern or relative arrangement.

In some embodiments, each one of the two portions is a single contiguous portion, as in devices 10 and 28. In some alternative embodiments, one or both of the two portions is made up of at least two physically-separate subportions.

In some embodiments, the dark-colored portion and light-colored portion border each other, as in device 10 and 28. In some alternative embodiments, the dark-colored portion and light-colored portion border are physically separated one from the other.

In some embodiments, one of the two portions encircles the other one of the two portions. In device 10 (FIG. 1), light-colored pollution-characterizing portion 24b encircles dark-colored hydration-characterizing portion 24a. In some related alternative embodiments, a dark-colored hydration-characterizing portion encircles a light-colored pollution-characterizing portion.

Device with Sebum-Characterizing Portion

As discussed in the introduction, it is known to press a hydrophobic microporous film (e.g., of microporous polypropylene) against the skin to absorb sebum from the skin. The normally opaque film becomes transparent in places where sebum is absorbed. Analysis of the microporous film allows characterization of the sebum gland activity (e.g., oily skin or not oily skin). Herein, such a sebum-absorbing hydrophobic microporous film is considered a sebum indicator.

In some embodiments, the teachings herein provide for a device having a thin body that has an upper surface and a lower surface (hereinfurther called the contact face). At least part of the contact face, called the adhesive-bearing portion, is covered with adhesive. The adhesive-bearing portion surrounds a sebum indicator, such as a sebum-absorbing hydrophobic microporous film, which changes appearance subsequent to absorbing sebum (e.g., changes from opaque to transparent) from skin.

For use, the contact face is pressed against a human skin surface (e.g., the skin of a forehead or cheek). In preferred embodiments, unlike prior art devices such as the Sebufix® SF16 discussed in the introduction which are held by a user against the face, with the device according to the teachings herein the adhesive-bearing portion of the contact face maintains the contact face and, particularly the sebum indicator, in intimate contact with the human skin for a period of time, typically for at least 5 seconds. During this time, sebum is absorbed into the pores of the sebum indicator.

When desired, the user removes the device from the skin (preferably by peeling off, as known in the art of adhesive bandages). The sebum indicator is then inspected in any suitable way, in preferred embodiments by capturing a digital image of the contact face followed by computerized analysis of the captured image.

Places where sebum is absorbed are clearly observable due to the change of appearance of the sebum indicator, indicating the level of oiliness of the skin. Further, it has been found that the use of the adhesive-bearing portion of the contact face to maintain the contact face in contact with the human skin is not only more convenient but in some embodiments produces substantially clearer borders between places on the sebum indicator where sebum is absorbed and places on the sebum indicator where sebum is not absorbed. Such embodiments allow more accurate characterization of the skin, for example, relatively accurate pore size estimate and identification of wrinkles and scars. Without wishing to be held to any one theory, it is currently believed that the adhesive-bearing portion keeps the sebum indicator against the skin without substantial lateral movement, thereby preventing “smearing”. Additionally or alternatively, it is currently believed that the use of an adhesive to keep the sebum indicator in contact with the skin allows for maintaining the device against the skin with a repeatable pressure that is sufficient and not too great, providing more accurate results.

Accordingly, inspection of the contact face of a device comprising sebum indicator, especially computerized analysis of a digital image of the contact face, in some embodiments allows characterization of the oiliness of the skin (as known in the art) and/or features such as pore size and the presence of scars and wrinkles.

Thus, according to an aspect of some embodiments of the teachings herein, there is provided a device suitable for characterizing skin, comprising:
a body having an upper surface and a lower contact face;
the upper surface and the lower contact face both having an area of not less than 0.2 cm² (equivalent to 0.5 cm diameter circle) and not more than 1020 cm² (equivalent to a 18 cm diameter circle);
the body having a thickness of not less than 30 micrometers and not more than 10 mm, wherein at least a part of the contact face comprises an adhesive-bearing portion that surrounds the surface of a sebum indicator which changes appearance subsequent to absorbing sebum from skin.

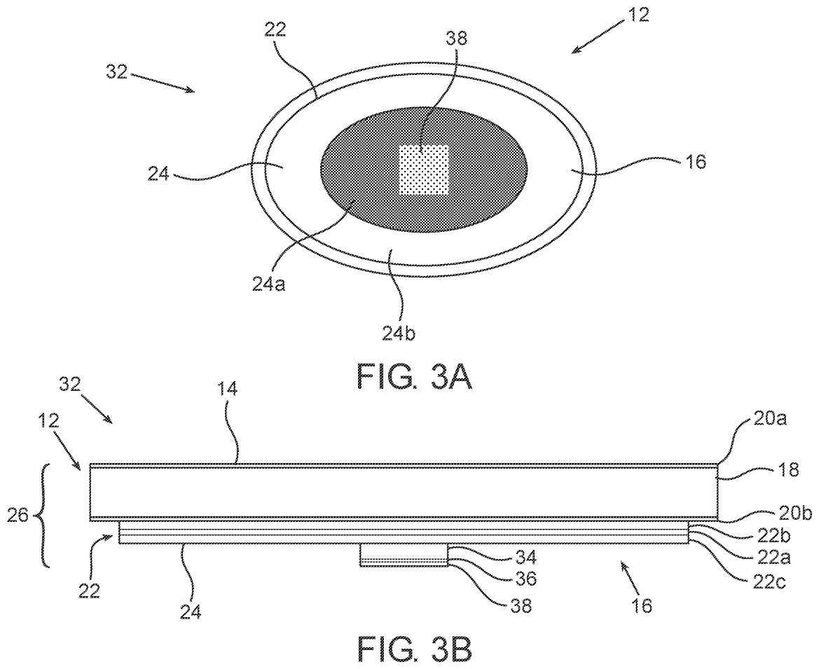

An exemplary embodiment of the device according to the teachings herein, device 32, is schematically depicted in FIG. 3A (bottom view without a protective cover) and FIG. 3B (side cross section). In FIG. 3B, the relative vertical sizes and the vertical scale of the components is distorted so that details of the layers making up the body of device can be seen. As with device 10 (FIG. 1) and device 28 (FIG. 2), device 32 is ordinarily provided to a user with a protective cover on the contact surface. Since such a protective cover is substantially similar to the protective cover of devices 10 and 28, it is not discussed in greater detail for the sake of brevity. Similarly, device 32 is substantially similar to devices 10 and 28 in top view so it is not discussed in greater detail for the sake of brevity. Device 32 includes a body 12, an upper surface 14 and a lower contact face 16.

Body 12 comprises a 250 micrometer thick polycarbonate (Lexan®) supporting sheet 18, oval-shaped with dimensions 5 cm×3 cm having a surface area of 11.8 cm².

The top side of supporting sheet 18 is entirely coated with a 20 micrometer thick layer of non-glare ink 20a, similarly to device 10.

The bottom side of supporting sheet 18 is entirely coated with a 20 micrometer thick layer of non-glare ink 20b, similarly to device 10. As with device 10, ink layer 20b on the bottom side of supporting sheet 18 includes a central oval of black ink surrounded by an oval ring of white ink.

Attached to the surface of ink layer 20b on the bottom side of supporting sheet 18 is an 4.5 cm×2.5 cm (8.8 cm² surface area) oval of double-sided adhesive film 22 (FIG. 3). Adhesive film 22 is 145 micrometer thick MED 6323 by Avery-Dennison having adhesive layers 22b and 22c. Adhesive layer 22b secures adhesive film 22 to ink layer 20b.

Attached to the center of adhesive film 22 through adhesive layer 22c is a 8 mm×8 mm square sheet of 75 micrometer thick black-colored polycarbonate (Lexan®), vertical spacer 34 (FIG. 3).

Attached to vertical spacer 34 is an 8 mm×8 mm square of 15 micrometer thick acrylic adhesive layer 36 and which is devoid of hydrophobic solvents that can be absorbed into the pores of a sebum-absorbing hydrophobic microporous film.

Attached to adhesive layer 36 is an 8 mm×8 mm square of 35 micrometer thick sebum-absorbing hydrophobic microporous film (such as Nexcare Oil Control Film Blotting Paper by 3M, Saint Paul, Minnesota, US) as a sebum indicator 38.

Vertical spacer 34, adhesive layer 36 and the microporous film of sebum indicator 38 together constitute a 125 micrometer thick microporous film assembly of device 32.

The portion of adhesive layer 22c that is not covered by the microporous film assembly constitutes an adhesive-bearing portion 24 of contact face 16 that surrounds the surface of sebum indicator 38.

Since adhesive film 22 in device 32 is transparent, ink layer 20b is clearly seen through contact face 16. When viewed from perpendicular towards contact face 16 (as in FIG. 3A) a central oval part of adhesive-bearing portion 24 that immediately surrounds the surface of microporous film 38 is dark-colored (black) and thereby constitutes a hydration-characterizing portion 24a of contact face 16 as discussed hereinabove which is in turn surrounded by an oval ring which is light-colored (white) and thereby constitutes a pollution-characterizing portion 24b of contact face 16 as discussed hereinabove.

A thickness 26 of body 12 of device 32 is 560 micrometers between upper surface 14 and the surface of sebum indicator 38 (which makes up 6.5% of the surface area of contact face 16) and 435 micrometers between upper surface 14 and adhesive-bearing portion 24 (which makes up 93.5% of the surface area of contact face 16).

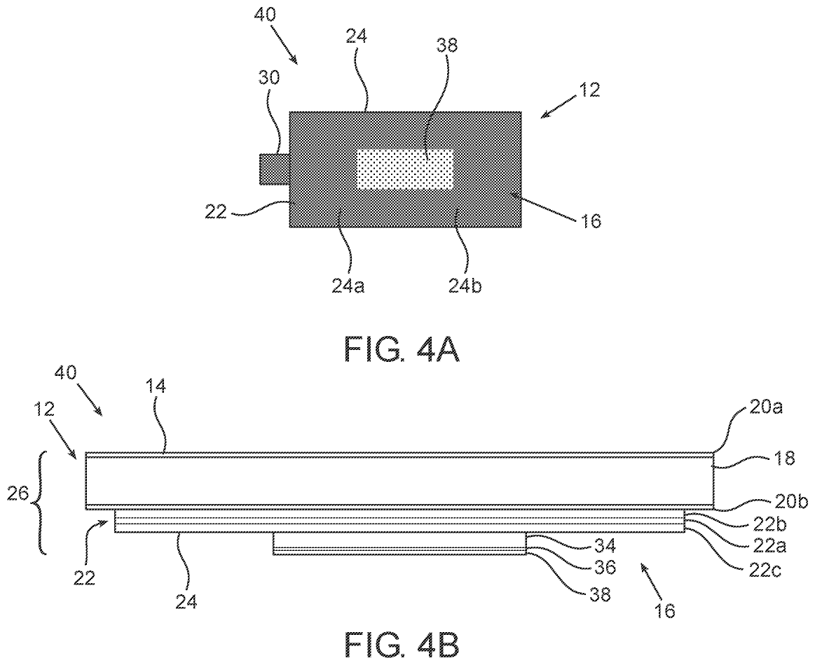

An additional exemplary embodiment of the device according to the teachings herein, device 40, is schematically depicted in FIG. 4A (bottom view without a protective cover) and FIG. 4B (side cross section). In FIG. 4B, the relative vertical sizes and the vertical scale of the components is distorted so that details of the layers making up the body of device can be seen. As with device 10 (FIG. 1) and device 28 (FIG. 2), device 40 is ordinarily provided to a user with a protective cover on the contact surface. Since such a protective cover is substantially similar to the protective cover of devices 10 and 28, it is not discussed in greater detail for the sake of brevity. Similarly, device 40 is substantially similar to devices 10 and 28 in top view so it is not discussed in greater detail for the sake of brevity. Device 40 includes a body 12, an upper surface 14 and a lower contact face 16. Body 12 comprises a 250 micrometer thick polycarbonate (Lexan®) supporting sheet 18, rectangle-shaped with dimensions 4 cm×2 cm having a surface area of 8 cm² with a 0.5 cm×0.5 cm tab 30 extending from one of the narrow sides.

As with device 28, in device 40 the top side of supporting sheet 18 is entirely coated with a 20 micrometer thick layer of non-glare ink 20a and the bottom side of supporting sheet 18 is entirely coated with a 20 micrometer thick layer of non-glare ink 20b. Ink layer 20b on the bottom side of supporting sheet 18 is uniformly black ink.

Attached to the surface of ink layer 20b on the bottom side of supporting sheet 18 is an 4 cm×2 cm rectangle of double-sided adhesive film 22 that entirely covers the bottom side except for at tab 30. Adhesive film 22 is 145 micrometer thick MED 6323 by Avery-Dennison having adhesive layers 22b and 22c. Adhesive layer 22b secures adhesive film 22 to ink layer 20b.

Attached to the center of adhesive film 22 through adhesive layer 22c is a 2.5 cm×1 cm rectangular sheet of 100 micrometer thick black-colored polycarbonate (Lexan®), vertical spacer 34.

Attached to vertical spacer 34 is a 2.5 cm×1 cm rectangle of 25 micrometer thick acrylic adhesive layer 36 and which is devoid of hydrophobic solvents that can be absorbed into the pores of a sebum-absorbing hydrophobic microporous film.

Attached to adhesive film 36 is a a 2.5 cm×1 cm rectangle of 35 micrometer thick sebum-absorbing hydrophobic microporous film (such as Nexcare Oil Control Film Blotting Paper by 3M, Saint Paul, Minnesota, US) as a sebum indicator 38.

Vertical spacer 34, adhesive layer 36 and the microporous film of sebum indicator 38 together constitute a 125 micrometer thick microporous film assembly of device 40.

The portion of adhesive layer 22c that is not covered by the microporous film assembly constitutes an adhesive-bearing portion 24 of contact face 16 that surrounds the surface of sebum indicator 38.

Since adhesive film 22 in device 40 is transparent, ink layer 20b is clearly seen through contact face 16. When viewed from perpendicular towards contact face 16 (as in FIG. 4A) the entirety of adhesive-bearing portion 24 is dark-colored (black) and thereby constitutes a hydration-characterizing portion 24a of contact face 16 as discussed hereinabove. In a non-depicted related embodiment, the entire underlying ink layer and the entire adhesive-bearing portion is light-colored (white), and thereby constitutes a pollution-characterizing portion of the contact face as discussed hereinabove. In embodiments analogous to device 40, the ink layer analogous to ink layer 22b comprises both a dark-colored ink portion that constitutes a hydration-characterizing portion and a light-colored ink portion that constitutes a pollution-characterizing portion.

A thickness 26 of body 12 of device 40 is 595 micrometers between upper surface 14 and the surface of sebum indicator 38 (which makes up 29% of the surface area of contact face 16) and 435 micrometers between upper surface 14 and adhesive-bearing portion 24 (which makes up 71% of the surface area of contact face 16). The thickness of the microporous film assembly is 160 micrometers.

### Sebum Indicator

As noted above, in some embodiments at least a part of the contact face of a device comprises an adhesive-bearing portion that surrounds the surface of a sebum indicator. A sebum indicator is a portion of the contact face which changes appearance subsequent to absorbing sebum from skin. As known in the art and discussed in the introduction, in some embodiments the sebum indicator is a hydrophobic microporous film such as microporous polypropylene. Suitable sebum indicators also include hydrophobic microporous films which are described in U.S. Pat. Nos. 4,532,937, 5,119,828, and 5,935,521 which are included by reference as if fully set forth herein to provide literal enabling support for a sebum indicator. Such a microporous film is secured to other components of the device in any suitable fashion, for example with a suitable adhesive or ultrasonic welding.

The thickness of the sebum-absorbing hydrophobic microporous film is any suitable thickness. In some embodiments, the thickness of such a sebum-absorbing hydrophobic microporous film is between about 10 micrometers and about 60 micrometers. In some embodiments, the thickness is not less than about 12 micrometers. Additionally or alternatively, in some embodiments, the thickness is not more than about 45 micrometers.

The porosity of the sebum-absorbing hydrophobic microporous film is any suitable porosity. In some embodiments, the porosity of such a sebum-absorbing hydrophobic microporous film is between about 30% and about 70%. In some embodiments, the porosity is not less than about 35%. Additionally or alternatively, in some embodiments, the porosity is not more than about 65%.

The average pore size of the sebum-absorbing hydrophobic microporous film is any suitable average pore size. In some embodiments, the average pore size of such a sebum-absorbing hydrophobic microporous film is between about 0.01 micrometers and about 0.1 micrometers. In some embodiments, the average pore size is not less than about 0.02 micrometers. Additionally or alternatively, in some embodiments, the average pore size is not more than about 0.08 micrometers.

The air permeability of the sebum-absorbing hydrophobic microporous film is any suitable air permeability. In some embodiments, the air permeability of such a sebum-absorbing hydrophobic microporous film is between about 50 Gurley seconds and about 1000 Gurley seconds. In some embodiments, the air permeability is not less than about 100 Gurley seconds. Additionally or alternatively, in some embodiments, the air permeability is not more than about 900 Gurley seconds.

In preferred embodiments, a sebum indicator comprises or is a hydrophobic open-celled microporous polymer film (e.g., microporous polypropylene) which is opaque to light when the pores are filled with air and which is substantially translucent when the film pores are filled with sebum. One particularly suitable such microporous film is a microporous polypropylene that is commercially available, for example, from Celgard LLC (Charlotte, North Carolina, USA), for example, Celgard® 2400. Other suitable hydrophobic open-celled microporous materials can include suitable nonwoven materials comprising fibers selected from the group consisting of polyester, polyether and polyolefin fibers, for example, nonwoven pulp sheets impregnated with polyethylene.

As noted above, in some embodiments, places on a sebum indicator that absorb sebum from skin change appearance, for example, from opaque to transparent. In some embodiments, to assist in identifying what part of a sebum indicator has absorbed sebum and what part has not absorbed sebum, the portion of the body of the device underlying the sebum indicator is not light-transparent. In some such embodiments, the portion of the body has a distinct color (e.g., bright yellow) and/or has distinct optical properties, for example is reflective (mirrored).

Preferably, the sebum indicator is present on the contact face as a flat surface without irregularities, allowing the entire surface of the sebum indicator to make contact with the skin.

The size of the sebum indicator on the contact surface is any suitable size. In preferred embodiments, the sebum indicator makes up between about 5% and about 50% of the surface area of the contact face. In some preferred embodiments, the sebum indicator has a surface area of not less than 0.25 cm² (equivalent to a 0.5 cm×0.5 cm square). In some preferred embodiments, the sebum indicator has a surface area of not more than 36 cm² (equivalent to a 6 cm×6 cm square).

The sebum indicator is of any suitable shape and is arranged in any suitable manner on the contact face.

### Single-Type of Sebum-Absorbing Hydrophobic Microporous Film

In some embodiments, a device includes a sebum indicator comprising a single type of hydrophobic microporous film on the contact face.

In some such embodiments, the microporous film is arranged on the contact surface in a single contiguous area, e.g., as a single sheet.

Alternatively, in some embodiments, the microporous film is arranged on the contact surface as at least two physically separate sheets, separated by a portion of the contact face that is not a microporous film. In preferred such embodiments, the sheets are separated by an adhesive-bearing portion of the contact face.

### Multiple-Types of Sebum-Absorbing Hydrophobic Microporous Film

While developing the device, the Inventors discovered that when different types of microporous films are contacted with a skin surface, substantially different patterns of sebum absorption are found on the respective surfaces.

For example, some microporous films provide a rough sebum-absorption pattern that is suitable for providing a qualitative characterization of the oiliness of the skin (more oily/less oily) as well as the presence of relatively large wrinkles and blemishes.

Other microporous films provide a fine sebum-absorption pattern that is suitable for providing information such as the number, size and size distribution of skin pores as well as the presence of fine wrinkles. Although not wishing to be held to any one theory, it is currently believe that these different patterns of sebum absorption are a result of different characteristics such as different film thickness, different average pore size, different porosity and/or different air permeability. For example, generally speaking thicker (greater than about 25 micrometers) films yield rough sebum-absorption patterns while thinner (less than about 20 micrometers) films yield fine sebum-absorption patterns.

Accordingly, in some embodiments, a device includes a sebum indicator comprising at least two different types of sebum-absorbing hydrophobic microporous film on the contact face, each different type of microporous film having different sebum-absorbing properties. Such embodiments allow a single device to provide different types of information about the skin from each one of the different types of microporous film. In some embodiments, a device includes a sebum indicator comprising a number of different types of microporous films, the number greater than 1, for example, 2, 3, 4, 5, 6 and even more than 6.

In some such embodiments, the sebum indicator comprises two different types of microporous film that are arranged on the contact surface in a single contiguous area, e.g., as two adjacent sheets with substantially no separation therebetween.

Alternatively, in some embodiments, the sebum indicator comprises two different types of microporous film that are arranged on the contact surface so as to be separated by a portion of the contact face that is not a microporous film. In preferred such embodiments, the two microporous films are separated by an adhesive-bearing portion of the contact face.

### Vertical Spacer

The microporous film or films that constitute the sebum indicator are attached to other components of the device in any suitable way. In some embodiments, the sebum indicator comprises a microporous film assembly comprising: a vertical spacer having a front side and a back side, the microporous film is attached to the front side of the vertical spacer; and the microporous film assembly is attached to other components of the device through the back side of the vertical spacer.

A vertical spacer is a thin planar component having a front side and a back side.

A microporous film that constitutes at least part of the sebum indicator is attached to a front side of the vertical spacer in any suitable way, in some embodiments using an adhesive. The adhesive is preferably an adhesive, such as an acrylic adhesive, that is devoid of solvent or other material that can leach into the microporous film.

Preferably, the adhesive layer is as thin as possible, typically having a thickness of between about 15 micrometers and about 30 micrometers. In some embodiments, the microporous film is attached to the vertical spacer with double-sided adhesive film.

The back side of the vertical spacer is attached to other components of the device in any suitable way so as to maintain the microporous film on the contact face of the device. As discussed with reference to device 32 (FIG. 3) and device 40 (FIG. 4), in some preferred embodiments, the device comprises an adhesive layer which constitutes the adhesive-bearing portion of the contact face of the device and also contacts the back side of the vertical spacer so as to maintain the microporous film on the contact face of the device.

The vertical spacer is made of any suitable material having any suitable properties. In preferred embodiments, the vertical spacer is stiff and self-supporting, that is to say, that when the device is held perpendicular to the gravity vector by one end, the body sags by no more than 10% and more preferably by no more than 5% as a result of gravity. In some preferred embodiments, the vertical spacer is elastic and ordinarily flat, that is to say, when unconstrained by an external force reverts from a curved shape to a flat shape. Preferably, the vertical spacer is not stretchy. The vertical spacer is of any suitable thickness, but is preferably as thin as possible so that a microporous film supported on a vertical spacer does not excessively protrude from the surrounding adhesive-bearing portion of the contact face. In some embodiments, the vertical spacer has a thickness of between about 50 micrometers and about 250 micrometers. In some such embodiments, the vertical spacer has a thickness of not less than about 75 micrometers and/or a thickness of not more than about 125 micrometers. The vertical spacer is made of any suitable material. It has been found that a particularly suitable material for a vertical spacer is a polycarbonate such as Lexan®.

A challenge when using two different types of sebum-absorbing hydrophobic microporous films for a sebum indicator occurs when the thickness of the two microporous films is different. In some such embodiments, the sebum indicator comprises two different microporous film assemblies:
a first microporous film assembly having a first microporous film with a first thickness attached to a first vertical spacer having a thickness; and
a second microporous film assembly having a second microporous film with a second thickness attached to a second vertical spacer having a thickness,
   wherein the thickness of the first microporous film assembly and the thickness of the second microporous film assembly are within 5 micrometers of each other, and in some embodiments are within 3 micrometers of each other.

In some embodiments, the first microporous film is attached to the first vertical spacer with a first adhesive layer and the second microporous film is attached to the second vertical spacer with a second adhesive layer.

In some embodiments, the first vertical spacer and the second vertical spacer have a different thickness. Additionally or alternatively, in some embodiments, the first microporous film is attached to the first vertical spacer with a first adhesive layer, the second microporous film is attached to the second vertical spacer with a second adhesive layer, and the thicknesses of the first and second adhesive layers are different.

As a result, the two different microporous films protrude from the adhesive-bearing portion of the contact face by approximately the same amount, in some embodiments within 5 micrometers and even within 3 micrometers.

For example, such a device may have:
a 16 micrometer thick first microporous film attached to a 120 micrometer thick first vertical spacer with a 102 micrometer thick double-sided adhesive film constituting a 238 micrometer thick first microporous film assembly; and
a 35 micrometer thick second microporous film attached to a 100 micrometer thick second vertical spacer with a 102 micrometer thick double-sided adhesive film constituting a 237 micrometer thick second microporous film assembly

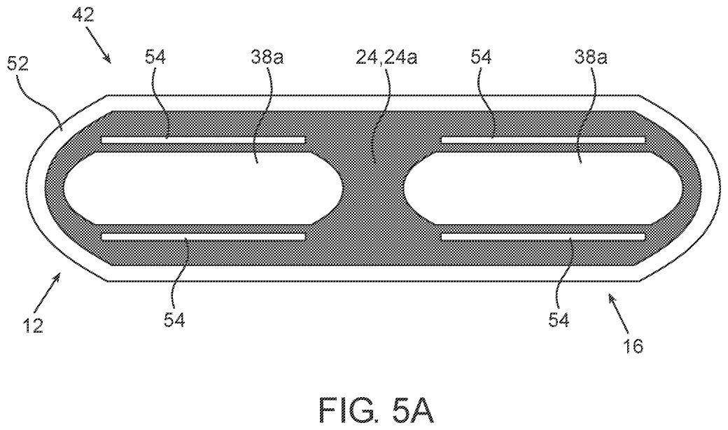

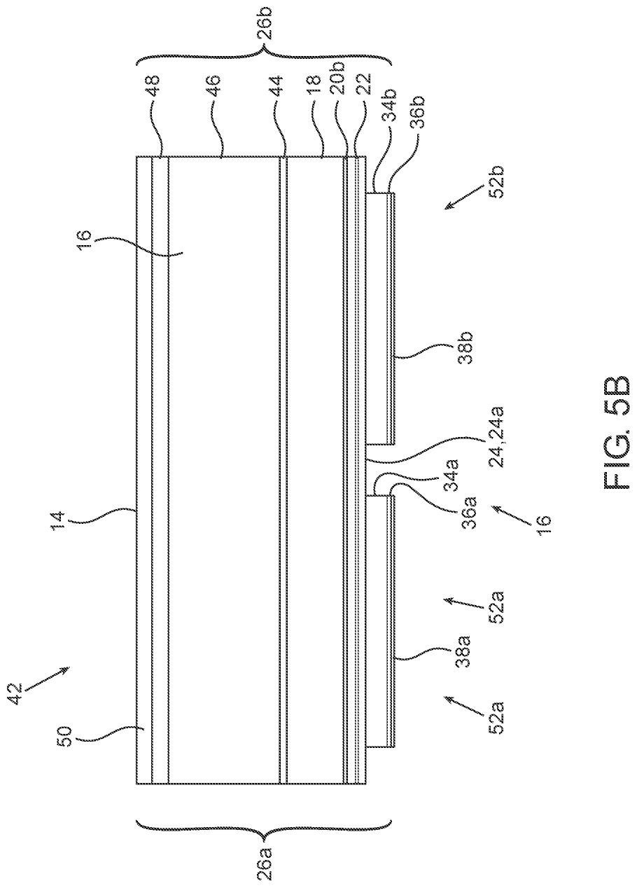

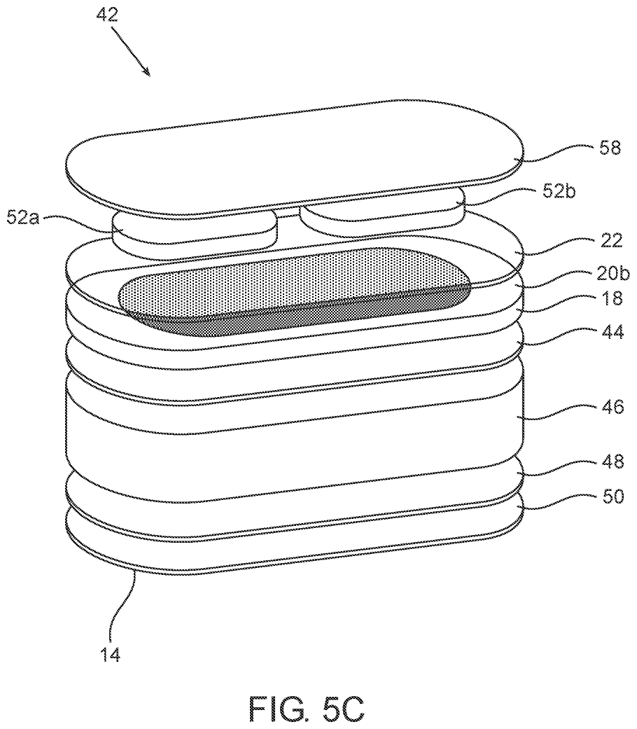

An additional exemplary embodiment of the device according to the teachings herein, device 42, is schematically depicted in FIG. 5A (bottom view without a protective cover), FIG. 5B (side cross section) and FIG. 5C (schematic exploded view). In FIG. 5B, the relative vertical sizes and the vertical scale of the components is distorted so that details of the layers making up the body of device can be seen. Device 42 is substantially similar to device 10 (FIG. 1) and device 28 (FIG. 2) in top view so it is not discussed in greater detail for the sake of brevity.

Device 42 includes a body 12, an upper surface 14 and a lower contact face 16. Body 12 comprises a 250 micrometer thick polycarbonate (Lexan®) supporting sheet 18, having a pointed-end rectangle-shape with dimensions 48 mm×18 mm having a surface area of 650 mm².

Attached to the upper surface of supporting sheet 18 with a 30 micrometer thick acrylic adhesive layer 44 is a 2000 micrometer thick elastically-compressible foam pressure-distribution layer 46.

Attached to the entire upper surface of pressure-distribution layer 46 is a 133 micrometer thick printed metallized polyester with adhesive 48 that includes printed on the upper surface thereof an image-correction feature, an identification mark and decorative features.

Attached to the entire upper surface of metallized polyester 48 is a 40 micrometer thick matte transparent polyester film with adhesive 50 that renders upper surface 14 of device 42 non-glare.

The lower surface of supporting sheet 18 is entirely coated with a 20 micrometer thick layer of non-glare ink 20b. Ink layer 20b on the bottom side of supporting sheet 18 includes a 1.5 mm wide white periphery 52 having a total area of 168 mm², four white lines (labelled 54) each 9 mm long and being about 0.1 mm wide, the balance being black. In similar embodiments, such lines are even thinner, typically being not less than about 0.05 mm and not more than about 0.1 mm wide.

Attached to the surface of ink layer 20b is a 102 micrometer thick double-sided adhesive film 22 (MED 1832) that entirely covers ink layer 20b.

Along the center line of body 12 are two 75 micrometer thick pointed-end rectangular sheets of black colored polycarbonate (Lexan®) constituting vertical spacers 34a and 34b. Vertical spacers 34a and 34b are separated by a portion of adhesive film 22.

Attached to vertical spacers 34a and 34b and entirely covering the upper surface thereof are 20 micrometer thick layers of acrylic adhesive 36a and 36b which is devoid of hydrophobic solvents that can be absorbed into the pores of a sebum-absorbing hydrophobic microporous film.

Attached to adhesive layer 36a and completely covering the upper surface thereof is 35 micrometer thick sebum-absorbing hydrophobic microporous film 38a (such as Nexcare Oil Control Film Blotting Paper by 3M, Saint Paul, Minnesota, USA) as a first portion of sebum indicator 38. Together, vertical spacer 34a, adhesive layer 36a and microporous film 38a constitute a first 130 micrometer thick microporous film assembly 52a of device 42.

Attached to adhesive layer 36b and completely covering the upper surface thereof is 16 micrometer thick sebum-absorbing hydrophobic microporous film as a second portion 38b of sebum indicator 38. Together, vertical spacer 34b, adhesive layer 36b and microporous film 38b constitute a second 111 micrometer thick microporous film assembly 52b of device 42. In some alternative embodiments, vertical spacer 34b is 100 micrometers thick so that second microporous film assembly 52b is 136 micrometers thick, only 6 micrometers thicker than first microporous film assembly 52a. In some alternative embodiments, vertical spacer 34b is 95 micrometers thick so that second microporous film assembly 52b is 131 micrometers thick, only 1 micrometer thicker than first microporous film assembly 52a.

Microporous film 38a and microporous film 38b together constitute a sebum indicator of device 42 (see FIG. 5).

The surface area of each one of the microporous films 38a and 38b is 145 mm² so that sebum indicator 38 has a total surface area of 290 mm². As a result, contact face 16 has a total surface area of 796 mm², of which white periphery 52 is 21% (168 mm²), sebum indicator 38 is 36% (290 mm²) and the black area that constitutes a hydration-characterizing portion of device 42 is a little less than the balance of 43%. The portion of adhesive film 22 that is not covered by sebum indicator 38 is the adhesive-bearing portion 24 of contact face 24 that surrounds the surfaces of sebum indicators 38 and makes up 64% of the surface area of contact face.

Since in device 42, depicted in FIGS. 5, adhesive film 22 is transparent, ink layer 20b is clearly seen through contact face 16. When viewed from perpendicular towards contact face 16, as in FIG. 5A, the entirety of adhesive-bearing portion 24 that is dark-colored (black) constitutes a hydration-characterizing portion 24a of contact face 16 as discussed hereinabove. In a non-depicted related embodiment, the entire underlying ink layer and the entire adhesive-bearing portion is light-colored (white), and thereby constitutes a pollution-characterizing portion of the contact face as discussed hereinabove.

A thickness 26a of body 12 of device 42 between upper surface 14 and the portion of contact face defined by microporous film assembly 52a is 2705 micrometers.

A thickness 26b of body 12 of device 42 between upper surface 14 and the portion of contact face defined by microporous film assembly 52b is 2686 micrometers.

A thickness of body 12 of device 42 between upper surface 14 and the portion of contact face defined by adhesive layer 22b is 2575 micrometers.

### Method of Attaching a Sebum Indicator to Contact Face

As noted above, in some embodiments, the adhesive used for implementing a pollution-characterizing portion and/or a hydration characterizing-portion of a device according to the teachings herein is also used to keep the device attached to the skin. In some such embodiments, this is advantageously done by using a double-sized adhesive film attached to a supporting sheet which, inter alia, helps provide the body of the device with the desired physical characteristics as discussed above. Additionally, in some such embodiments, the use of a specific adhesive layer (e.g., adhesive film 22) for multiple functions (a component of a pollution-characterizing portion, a component of a hydration-characterizing portion and/or an adhesive for attached the device to the skin) makes the device easier to use and to manufacture.

In some embodiments, at least a part of the contact face comprises an adhesive-bearing portion that surrounds the surface of a sebum indicator. A challenge in practically implementing a device of the teachings herein which includes a sebum indicator is attaching a sebum indicator (typically a very-thin, fragile, tissue-like sheet) to the contact face. In some embodiments, the contact face comprises a layer of adhesive to which a sebum indicator is attached. As a result, the same adhesive that surrounds the surface of the sebum indicator is the adhesive that keeps the sebum indicator attached to the contact face.

An alternative method depicted with reference to device 42 depicted in FIG. 5 and noted above comprises separately making a microporous film assembly comprising a vertical spacer to which a microporous film is attached, for example, using an adhesive. According to an aspect of some embodiments of the teachings herein there is provided a method that is particularly suitable for making a microporous film assembly for use as a sebum indicator comprising:
providing a sheet of material suitable for being a vertical spacer having a front side and a back side;
covering the front side with a layer of adhesive;
adhering to the layer of adhesive covering the front side a sheet of sebum-absorbing microporous film to make a laminated workpiece; and
cutting the laminated workpiece to make a microporous film assembly.

Preferably, the adhesive layer is as thin as possible, typically having a thickness of between about 15 micrometers and about 30 micrometers. In some embodiments, the adhesive layer is a single adhesive layer that is disposed between the front side of the sheet of material and the sheet of sebum-absorbing microporous film. In some alternate embodiments, the adhesive layer is a component of a double-sided adhesive film. As noted above, the adhesive is preferably an adhesive devoid of hydrophobic solvents that are absorbed into the microporous film (e.g., an acrylic adhesive).

The sheet of material is of any suitable material, having one, more than one, and preferably all of the properties of a vertical spacer as discussed above and which are not repeated for the sake of brevity. A particularly suitable material is a polycarbonate such as Lexan®.

The sheet of material is of any suitable thickness, preferably between about 50 micrometers and about 250 micrometers, more preferably up to about 125 micrometers.

As noted above, in some embodiments, a microporous film that functions as a sebum indicator is ordinarily opaque so, when viewed from perpendicular to the film surface has a color that corresponds to that of the film. After contact with a skin surface, portions of the microporous film that absorb sebum become transparent, so when viewed from perpendicular to the film surface the portions have the color of the underlying material which, for microporous film assembly made according to the method herein, is the color of the vertical spacer. Accordingly, to aid in identifying what portions of a sebum indicator have absorbed sebum, in preferred embodiments the sheet of material has a color that is substantially different from the color of the microporous film attached thereto.

Cutting the laminated workpiece to make a microporous film assembly is done in any suitable manner, for example die cutting or laser cutting.

In some particularly preferred embodiments, the sheet of material suitable for being a vertical spacer, the adhesive and the microporous film are all provided as rolls. Such rolls allow for particular efficient manner of making a laminated workpiece that can be cut to make a microporous film assembly. For example, in some embodiments, a roll of each one of the three components is mounted on a spindle of a continuous laminator machine. The material suitable for being a vertical spacer is fed from the first spindle. The adhesive is fed from the second spindle with continuous detachment of the bottom protective liner of the adhesive roll, exposing the lower surface of the adhesive layer which contacted with the upper surface of material suitable for being a vertical spacer and then passed under a first pressure roller. Upon exiting the pressure roller, the upper protective liner of the adhesive roll is removed, exposing the upper surface of the adhesive layer. The microporous film is fed from the third spindle and contacted with the exposed upper surface of the adhesive later and then passed under a second pressure roller, thereby providing the laminated workpiece that can be cut to any desired sized.

The method allows for simultaneously making many microporous film assemblies, having any desired thickness and dimensions. The resulting microporous film assembly are non-sticky, one side being the back side of the incipient vertical spacer, the other side being the microporous film. The microporous film assemblies are preferably relatively stiff, tough and flat (as a result of the properties of the vertical spacer) so are easy to manipulate with robotic machinery that assembles a device.

A device comprising a sebum indicator according to the teachings herein can be made in any suitable method. According to an aspect of some embodiments of the teachings herein there is also provided a method for making a device comprising a sebum indicator as described herein, the method comprising:
a. providing a sheet of material suitable for being a supporting sheet of a body of the device having a front side and a back side;
b. providing a microporous film assembly comprising a vertical spacer having a front side and a back side, with a microporous film attached to the front side of the vertical spacer;
c. covering the front side of the sheet of material with a layer of adhesive;
d. contacting the back side of the vertical spacer of the microporous film assembly with the layer of adhesive covering the front side of the sheet of material where the microporous film assembly is surrounded by a surface of the layer of adhesive, thereby attaching the microporous film assembly to the front side of the sheet of material
e. cutting the sheet of material to yield an incipient device comprising a sebum indicator, wherein the cutting is such that a microporous film assembly constituting an incipient sebum indicator of a device is surrounded by a surface of the layer of adhesive constituting an adhesive-bearing portion of a device.

The adhesive layer is of any suitable adhesive having properties required from the adhesive-bearing portion of a device including sufficient adherence to the skin of a human. In some embodiments, the adhesive layer is a single adhesive layer that is disposed between the front side of the sheet of material and the microporous film assembly. In some preferred embodiments, the layer of adhesive is a component of a double-sided adhesive film as discussed hereinabove.

The sheet of material is of any suitable material, having one, more than one, and preferably all of the properties of a supporting sheet as discussed above and which are not repeated for the sake of brevity. A particularly suitable material is a polycarbonate such as Lexan®.

The sheet of material is of any suitable thickness, preferably between about 50 micrometers and about 450 micrometers, more preferably not less than about 120 micrometers.

The color of the sheet of material is any suitable color.

In some embodiments, the method is used to make a device having a contact face with a dark-colored part to serve as a hydration-characterizing portion of the contact face. In some such embodiments, the color of the sheet of material is chosen to be dark so that the entire adhesive-bearing portion of the contact face can serve as the hydration-characterizing portion.

Alternatively, in some embodiments, the method is used to make a device having a contact face with a light-colored part to serve as a pollution-characterizing portion of the contact face. In some such embodiments, the color of the sheet of material is chosen to be light so that the entire adhesive-bearing portion of the contact face can serve as the pollution-characterizing portion.

Alternatively, in some embodiments, the method is used to make a device having a contact face with a dark-colored part and/or a light-colored part to implement a hydration-characterizing portion of the contact face; and/or a pollution-characterizing portion of the contact face and/or to add a feature like an image correction feature, an identifier mark and/or a feature that assists in orientation identification. In some such embodiments the method further comprises, prior to ‘c’ (covering the front side of the sheet of material with a layer of adhesive) applying a colorant to the front side of the sheet of material having the desired color pattern. In some embodiments, the entire front side of the sheet of material is covered with a layer of colorant. In some embodiments, colorant is applied to only parts of the front side of the sheet of material. Subsequently, in ‘c’, the layer of adhesive is applied to contact the colorant. Any suitable colorant is used. In some preferred embodiments, a colorant such as ink is applied, for example by digital printing or screen printing. This allows application of a thin layer of colorant (typically less than 40 micrometers and even less than 30 micrometers) that adheres well to the sheet of material.

Cutting the sheet of material to yield an incipient device comprising a sebum indicator is done in any suitable manner, for example die cutting or laser cutting.

As with the method of making the sebum indicator described above, in some particularly preferred embodiments, some or all of the method is performed where the some or all of the components are provided as rolls of material and assembly is performed using a continuous laminator machine.

The method allows for concurrently and even simultaneously making many devices. A single sheet of material can be used to make devices having one, two or more sebum indicators. A single sheet of material can be used to make devices having two or more identical sebum indicators or devices having two or more different sebum indicators.

As discussed hereinabove, a device according to the teachings herein is preferably provided to a user with a protective cover. The protective cover is added to a device at any suitable stage of the making of the device. In some preferred embodiments, an incipient protective cover is added subsequent to attachment of the microporous film assembly but prior to the cutting. Accordingly, in some embodiments, the method further comprises, subsequent to ‘d’ and prior to ‘e’, covering the front side with an incipient protective cover which covers the microporous film assembly and adheres to the front the through the surface of adhesive surrounding the microporous film assembly.

As discussed above, in some embodiments, a device according to the teachings herein has one or more layers on the back side of the device (the side opposite the sebum indicator). Such layers are attached to the back side of the device in any suitable fashion. In some embodiments, such layers are attached to the back side of the sheet of material prior to ‘c’, where the front side of the sheet of material is covered with a layer of adhesive. Alternatively, in some embodiments, such layers are attached to the back side of the sheet of material subsequent to adding an incipient protective cover, but prior to ‘e’ the cutting of the sheet. Alternatively, in some embodiments, in ‘c’, the layer of adhesive is applied to the sheet of material with a cover (e.g., a liner of a double-sided adhesive film). Layers are attached to the back side of the sheet of material and subsequently the cover is removed, allowing attachment of the microporous film assembly.

Device with Two Different Types of Sebum-Absorbing Hydrophobic Microporous Films

Described above is a device with a contact face that comprises a sebum indicator that is surrounded by an adhesive-bearing portion of the contact face, the sebum indicator comprising at least two different types of the sebum-absorbing hydrophobic microporous film. In some embodiments, a device is provided with a contact face that comprises a sebum indicator, the sebum indicator comprising at least two different types of the sebum-absorbing hydrophobic microporous film, the sebum indicator not necessarily surrounded by an adhesive-bearing portion. Thus, according to an aspect of some embodiments of the teachings herein, there is also provided a device suitable for characterizing skin, comprising:
a body having an upper surface and a lower contact face;
the upper surface and the lower contact face both having an area of not less than 0.2 cm² (equivalent to 0.5 cm diameter circle) and not more than 1020 cm² (equivalent to a 18 cm diameter circle);
the body having a thickness of not less than 30 micrometers and not more than 10 mm, wherein at least a part of the contact face comprises a surface of a sebum indicator which changes appearance subsequent to absorbing sebum from skin, the sebum indicator comprising at least two different types of the sebum-absorbing hydrophobic microporous film on the contact face, each different type of microporous film having different sebum-absorbing properties.

In some such embodiments, the contact face is devoid of an adhesive-bearing portion. Alternatively, in some such embodiments, the contact face comprises an adhesive-bearing portion that does not surround the sebum indicator.

Other details of the device are the same (mutatis mutandis) as described with reference to other embodiments so are not explicitly discussed here for brevity.

### Details of Other Features of the Device

Discussed above is an embodiment of a device that includes a contact face with an adhesive portion having a dark-colored portion and a light-colored portion and also an embodiment of a device that includes a contact face with a sebum-absorbing microporous polymer.

As is apparent from the discussion above and the Figures, in some embodiments a device only includes a contact face with an adhesive portion having a dark-colored portion and a light-colored portion, in some embodiments a device only includes a contact face with a sebum-absorbing microporous polymer and in some embodiments a device includes a contact face having both an adhesive portion having a dark-colored portion and a light-colored portion and a sebum-absorbing microporous polymer.

Features, options and embodiments of a body of a device according to the teachings herein are discussed in detail with reference to the embodiment that includes a contact face with an adhesive portion having a dark-colored portion and a light-colored portion. Such features and embodiments are also applicable to embodiments that include a contact face having a sebum-absorbing microporous polymer but are not repeated here for the sake of brevity.

Features, options and embodiments of an adhesive-bearing portion of a device according to the teachings herein are discussed in detail with reference to the embodiment that includes a contact face with an adhesive portion having a dark-colored portion and a light-colored portion. Such features and embodiments are also applicable to embodiments that include a contact face having a sebum-absorbing microporous polymer in so far as these are applicable but are not repeated here for the sake of brevity.

### Pressure Distribution Layer

In some embodiments, a device according to the teachings herein includes a pressure-distribution layer between the top surface and the contact face. The pressure-distribution layer is configured to distribute pressure applied to the top surface, e.g., with a finger, over the contact face. The pressure-distribution layer is of any suitable material, typically an elastically-compressible material such as a foam or a rubber, and is of any suitable thickness, in some embodiments not less than 500 micrometers and not more than 5000 micrometers. Device 42 depicted in FIG. 5 comprises a pressure-distribution layer 46.

### Contact Face

The arrangement of the adhesive-bearing portion of the contact face of a device and of the sebum indicator of a device is any suitable arrangement, for example, as in device 32 in FIGS. 3, device 40 in FIG. 4 and device 42 in FIG. 5.

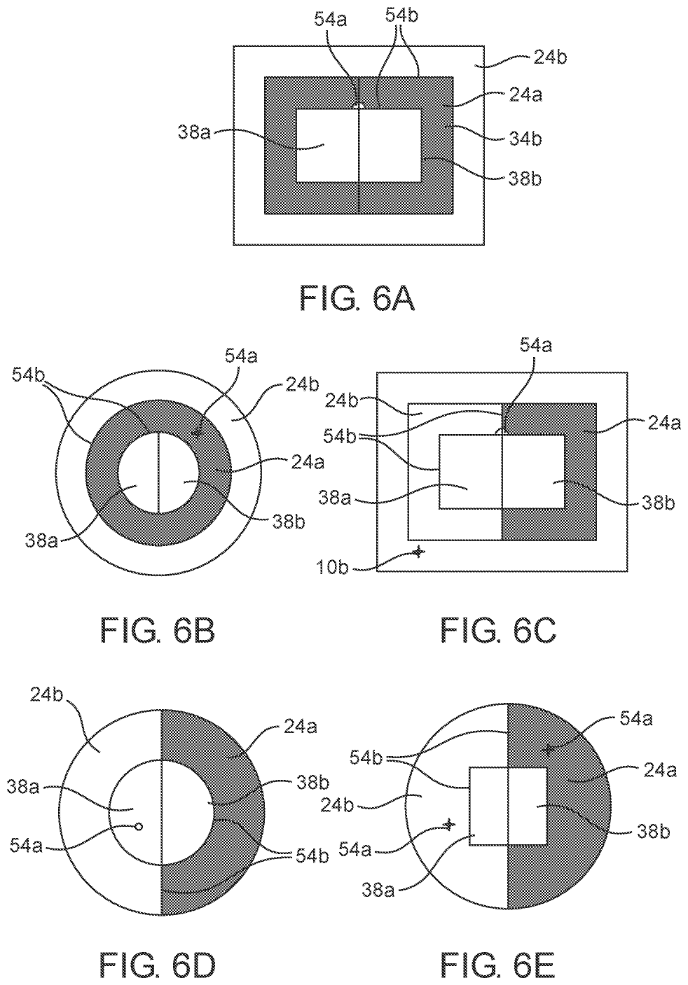

Additional non-limiting arrangements are depicted in FIGS. 6A-6E which show the contact face of five different embodiments. All the devices depicted in FIG. 6 have a adhesive-bearing portion with a dark part 24a (constituting a hydration-characterizing portion of the device) and a light part 24b (constituting a pollution-characterizing portion of the device). All the devices have a sebum indicator that includes two different sebum-absorbing hydrophobic microporous films 38a and 38b.

### Upper Surface

An upper surface of a device according to the teachings herein is any suitable upper surface. In preferred embodiments, the upper surface is configured to be photographed with a digital camera while adhering to the skin.

### Non-Glare Upper Surface

In preferred embodiments, at least some, preferably all, of the upper surface of a device is non-glare (e.g., matte) so when photographed with a flash, the upper surface does not specularly reflect light from the flash which reduces the quality of an acquired image. A non-glare upper surface can be provided in any suitable way, for example, by the selection of the material from which the upper surface is made and/or from applying a non-glare or matte coating to the upper surface. In device 10 (FIG. 1) and device 28 (FIG. 2), upper surface 14 is the surface of a non-glare ink layer 20a. In device 42 (FIG. 5) upper surface is the surface of matte transparent polyester film 50.

### Image-Correction Feature

As is known in the art, the appearance of an object in an acquired digital image, including the color and the white balance of the object in the image, is dependent on the lighting conditions during acquisition of the image.

In some embodiments, a user using the device acquires an image of the upper surface of the body of the device when the device is attached to the skin. It is known that the color of the skin and pigmented irregularities to which the device is attached in the image will be highly dependent on the lighting conditions. As a result, computer-determined skin color in an image is inaccurate.

In some embodiments, the intended use of a device according to the teachings herein is for a person to use the device at home and acquire an image of the device when attached to the skin using a commonly available device such as a smartphone or computer camera. As a result, images acquired during the expected use of the device will have different ambient light conditions, different camera-flash characteristics (different smartphones have different camera flashes with different spectra and intensities) and different sensor characteristics (the sensors of the cameras of different smartphones have different spectral sensitivities).

Accordingly, in some preferred embodiments, the upper surface of the body of the device comprises at least one image-correction feature that assists in computerized correction of the color and/or white balance of an acquired image in which the upper surface appears. A person having ordinary skill in the art of image processing is familiar with different types of image-correction features (which typically include areas of specific reference colors) on the upper surface. In some embodiments, an image-correction feature is similar to or a variant of a commercially-available camera color calibration chart and/or white balance card set. When such embodiments are used, an acquired image can be computer analyzed also to determine the real color of the skin in the image, thereby providing an additional objective characterization of the skin and also allowing identification of features such as redness and irritation.

Ink layer 20a that defines upper surface 14 of device 10 (FIG. 1) and device 28 (FIG. 2) includes a color/white balance chart as an image-correction feature chart 54 including multiple regions having different colors as known in the art of digital image color correction.

Further, in some instances, due to the nature of the lens of the camera, the orientation of the upper surface relative to the lens of the camera or the manner in which the device is adhered to the skin, an image of the upper surface is distorted. Accordingly, in some preferred embodiments, the upper surface of a device comprises at least one image-correction feature that assists in computerized correction of the distortion of the portion of an acquired image that corresponds to the upper surface.

### Identifier Mark

In some embodiments, a user using the device acquires an image of the upper surface of the body of the device when the device is attached to the skin. To aid in computerized image analysis of such an acquired image and help avoid data-entry errors, it is useful to be able to identify a specific device in an acquired image.

Accordingly, in some embodiments, the upper surface of a device includes an identifier mark, i.e., a marking allowing computerized identification of the specific device from other such devices from an acquired image of the upper surface of the body of the device. Any suitable identifier can be used including identifiers known in the art such as barcodes, QR codes and variants thereof. In some embodiments, a user is provided with one or more devices, the upper surface of the provided devices including an identifier that allows, inter alia, identification of the user.

Ink layer 20a that defines upper surface 14 of device 10 (FIG. 1) and device 28 (FIG. 2) includes a QR code as an identifier mark 56 (FIGS. 1C and 2C). Depending on the embodiment, such a QR code can include information such as the identity of the user and the manufacture batch number and date.

### Orientation Identification

In some embodiments, a user using the device acquires an image of the upper surface of the body of the device when the device is attached to the skin, for example, to the face.

In some preferred embodiments, the upper surface of a device is shaped and/or has markings that allow unambiguous identification of the orientation of the device in an acquired image. Specifically, in some embodiments the upper surface is shaped and/or has markings so that in an acquired image, the upper surface has C1 rotational symmetry.

When the upper surface of a device is shaped and/or has markings that allow unambiguous identification of the orientation of the device, a computer analyzing an image of the device when attached to the skin can determine the part of the adhesive-bearing portion of the contact face contacted that contacts any specific area of the skin.

A person having ordinary skill in the art is able to select a shape and/or markings of an upper surface to allow unambiguous identification of the orientation of the device in an acquired image. In some embodiments, one or more features used for orientation identification can also be used as an image-correction feature to assist in computerized correction of the distortion of the portion of an acquired image that corresponds to the upper surface.

Ink layer 20a that defines upper surface 14 of device 10 (FIG. 1) and device 28 (FIG. 2) includes an image-correction feature 54 and an identifier mark 56 that are asymmetrically disposed on upper surface 14 so that in an image, upper surface has C1 symmetry. A person having ordinary skill in the art of image analysis is able, without undue experimentation, to configure a computer to identify the orientation of such a device in an image.

### Additional Features of the Lower Contact Face

As discussed above, in some embodiments use of the device includes contacting the contact face with skin for a period of time, removing the device, acquiring an image of the contact face and then computer-analyzing the image of the contact face in order to characterize the skin. In some embodiments, the contact face is configured to be photographed with a digital camera, in some embodiments in a manner similar or identical to the configuration of the upper surface as discussed hereinabove.

Accordingly, in some preferred embodiments, at least some, preferably all, of the contact face of a device is non-glare (e.g., matte) so when photographed with a flash, the contact face does not specularly reflect light from the flash which reduces the quality of an acquired image. A non-glare contact face can be provided in any suitable way, for example, by the selection of the material from which the contact face is made.

In some embodiments, the contact face of a device comprises at least one image-correction feature that assists in computerized correction of the color and/or white balance of an acquired image as described above for the upper surface. The contact face of the devices depicted in FIG. 6 all include an image correction feature 54a that is configured to assist in computerized correction of the color and/or white balance of an acquired image.

In some embodiments, the distribution and amount of dark-colored pollution on a light-colored pollution-characterizing portion of a contact face is used to characterize the skin. Additionally or alternatively, in some embodiments, the distribution and amount of light-colored skin cells on a dark-colored hydration-characterizing portion of a contact face is used to characterize the skin. Additionally or alternatively, in some embodiments, the distribution and amount of the sebum-characterizing portion of a contact face that absorbed sebum are used to characterize the skin. As noted above and known in the art, in some instances, the portion of an image that corresponds to a contact face in an acquired image is distorted. Such distortion can lead to an inaccurate characterization of the skin. Accordingly, in some preferred embodiments, the contact face of a device comprises at least one image-correction feature (in some embodiments being a demarcation, e.g., between one or more areas or regions of the contact face) that assists in computerized correction of the distortion of the portion of an acquired image that corresponds to the contact face. For example, parallel lines in one or both directions. Ink layer 20b of device 42 (FIG. 5) includes four distinctly colored lines as such an image-correction feature 54. The four lines have a known thickness and length. Similarly, the contact face of the devices depicted in FIG. 6 all include an image correction feature 54b that are clearly visible demarcations on between different regions of the contact face and can be used to assist in computerized correction of distortion in an acquired image. A person having ordinary skill in the art is able to configure an image-processing computer to correct an image of contact face 16 of device 42 with reference to image-correction feature 54 or of an image of a contact face of a device such as depicted in FIG. 6 with referenced to an image-correction feature 54b.

To aid in computerized image analysis of an acquired image of a contact face and help avoid data-entry errors, it is useful to be able to identify a specific device in an acquired image. Accordingly in some embodiments, the contact face of a device includes an identifier mark, i.e., a marking allowing computerized identification of the specific device from other such devices from an acquired image of the contact face of the device. Any suitable identifier can be used, such as discussed with referenced to an identifier mark of an upper surface.

In some embodiments, a contact face of a device is shaped and/or has markings that allow unambiguous identification of the orientation of the device in an acquired image, as discussed with referenced to the upper surface. In some embodiments, one or more features used for orientation identification can also be used as an image-correction feature to assist in computerized correction of the distortion of the portion of an acquired image that corresponds to the upper surface. The contact face of the devices depicted in FIG. 6 all include at least one image correction feature 54a that are asymmetrically disposed on contact face and can therefore be used to assist in computerized identification of the orientation of the device in an acquired image

### Protective Cover

In preferred embodiments, a device according to the teachings herein further comprises a user-removable protective cover that covers at least a portion of the contact face and is easily removable by a user. Such a protective cover is similar or identical to the backing with which prior art adhesive bandages are provided. The protective cover is a type of release liner, a well-known component in the art which is used to prevent a sticky surface (such as the adhesive-bearing portion of the contact face) from prematurely adhering. As known in the art of release liners, in some embodiments, a release liner comprises silicone, e.g., comprises a silicone coating or is impregnated with silicone.

The protective cover preferably covers the entire contact face, although in some embodiments some of the contact face is not covered by the protective cover. The protective cover is preferably a thin material such as a coated paper, plastic or foil, such as cellophane, mylar, or a polyester such as PET. In some embodiments, the protective cover is a single piece, but in other embodiments the protective cover comprises two or more separate parts as known in the art of adhesive bandages. The size and shape of the protective cover is preferably sufficient to cover the entire contact face, to protect the components of the contact face from contamination. In some embodiments, the protective cover is held in place by electrostatic forces and/or by an adhesive, preferably an adhesive of one or more of the adhesive-bearing portions regions. In some embodiments, a part of the protective cover (e.g., as a tab or lip) is configured to allow easier removal of the protective cover from the contact face.

Device 10 is provided with a 2-piece 100 micrometer thick protective cover 58 of coated paper, see FIG. 1B. Devices 28 is provided with a 1-piece 100 micrometer thick protective cover 58 of coated paper, see FIG. 2B. Device 42 is provided with a 100 micron thick protective cover 58, see FIG. 5C. For use of the respective device, protective covers 58 are peeled away from a respective adhesive-bearing surface 24 as known in the art of adhesive bandages.

### Package

Additionally or alternatively to a protective cover, in some preferred embodiments, a device is provided to a consumer packaged in a user-openable sealed package such as a sealed envelope as known in the art of wound dressings and adhesive bandages.

In some embodiments, a protective cover is part of the package, as known in the art of adhesive bandages.

In some embodiments, each device is individually packed in an own package.

### Use of a Device

An embodiment of an exemplary use of an embodiment of the device is as follows:

In some embodiments, a device is received by a user in a sealed package such as a sealed envelope. The envelope is opened and the device removed therefrom. The protective cover is peeled away from the contact face of the device, exposing the contact face.

The device is placed on the surface of skin to be characterized so that the contact face makes intimate contact with the skin for a few seconds, in some embodiments the adhesive on the contact face keeping the device adhered to the skin. In some embodiments, the user presses the upper face of the device to increase the quality of the contact of the contact face with the skin. In some embodiments, the user places the device on the T-zone (area of the face including the forehead and the nasal bridge). In some embodiments, the user places the device on the U-zone (continuous area of the face including the cheeks and chin). In some embodiments, two different devices are placed on the skin surface, one on the T-zone and one on the U-zone.

In some embodiments, the user uses a camera such as found on a smartphone or computer to acquire an image of the skin surface and of the upper surface of the body of the device.

The device (or devices) is peeled away from the skin.

A device is placed upper surface-down on a surface/contact face-up such as on a table. A camera, such as a smartphone camera, is used to acquire an image of the contact face.

The acquired images are sent to an image analysis device, typically a computer configured (e.g., software configured) to analyze the acquired images. In some embodiments the computer is a component of a smartphone, such as a smartphone used to acquired the images. In some embodiments, the computer is a local computer such as a home desktop or laptop computer. In some embodiments, the computer is a remote computer (e.g., cloud) to which the images are sent, for example, sent by the Internet.

In some embodiments, with reference to the identifier mark, the image of the upper surface while the device is attached to the user is analyzed to identify the user, when the device was sent to the user and/or the lot number of the device.

In some embodiments, with reference to the image correction feature, the image of the upper surface while the device is attached to the user is analyzed to identify the color of the skin of the user (e.g., Fitzpatrick scale) and, in some embodiments, skin features like freckles, sun damage, pigmentation, stains, marks, acne and (age-related) blotchiness.

The image of the contact face is analyzed. Typically, at least the portions of the acquired image that correspond to the characterizing portions of the contact face are identified, scaled and shape-corrected with reference to an image-correction feature to account for distortions such as lens distortion and camera-angle distortions.

In some embodiments, the image analysis device calculates the cleanliness of the skin with reference to the portion of the image that corresponds to the pollution-characterizing-portion.

In some embodiments, the image analysis device calculates the hydration of the skin with reference to the portion of the image that corresponds to the hydration-characterizing portion.

In some embodiments, the image analysis device calculates the oiliness and/or other characteristics of the skin with reference to the portion of the image that corresponds to the sebum indicator.

With the calculated skin characteristics and, in some embodiments, to the geographic location of the person (for example, as recovered from the metadata of the images), suitable cosmetic preparations can be provided, recommended or made for the person whose skin was characterized.

In some embodiments, the user is provided (immediately on conclusion of the image-analysis), with a numerical value of the results from the image of the contact face of the device. In some embodiments, the user receives a table with numerical values relating to one or more skin characteristics, in some embodiments, separately for the T-zone and the U-zone.

In some embodiments, the device is used periodically to characterize the skin under different conditions, e.g., different seasons, different locations, different times of day, after different activities and the like.

Unless otherwise defined, all technical and scientific terms used herein have the same meaning as commonly understood by one of ordinary skill in the art to which the invention pertains. In case of conflict, the specification, including definitions, takes precedence.

As used herein, the terms “comprising”, “including”, “having” and grammatical variants thereof are to be taken as specifying the stated features, integers, steps or components but do not preclude the addition of one or more additional features, integers, steps, components or groups thereof.

As used herein, the indefinite articles “a” and “an” mean “at least one” or “one or more” unless the context clearly dictates otherwise.

As used herein, when a numerical value is preceded by the term “about”, the term “about” is intended to indicate +/−10%.

As used herein, a phrase in the form “A and/or B” means a selection from the group consisting of (A), (B) or (A and B). As used herein, a phrase in the form “at least one of A, B and C” means a selection from the group consisting of (A), (B), (C), (A and B), (A and C), (B and C) or (A and B and C).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Embodiments of methods and/or devices described herein may involve performing or completing selected tasks manually, automatically, or a combination thereof. Some methods and/or devices described herein are implemented with the use of components that comprise hardware, software, firmware or combinations thereof. In some embodiments, some components are general-purpose components such as general purpose computers and digital processors. In some embodiments, some components are dedicated or custom components such as circuits, integrated circuits or software.

For example, in some embodiments, some of an embodiment is implemented as a plurality of software instructions executed by a data processor, for example which is part of a general-purpose or custom computer. In some embodiments, the data processor or computer comprises volatile memory for storing instructions and/or data and/or a non-volatile storage, for example, a magnetic hard-disk and/or removable media, for storing instructions and/or data. In some embodiments, implementation includes a network connection. In some embodiments, implementation includes a user interface, generally comprising one or more of input devices (e.g., allowing input of commands and/or parameters) and output devices (e.g., allowing reporting parameters of operation and results).

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

1. A device suitable for characterizing skin, comprising:
a body having an upper surface and a lower contact face;
said upper surface and said lower contact face both having an area of not less than 0.2 cm² (equivalent to 0.5 cm diameter circle) and not more than 1020 cm² (equivalent to a 18 cm diameter circle);
said body having a thickness of not less than 30 micrometers and not more than 10 mm, wherein at least a part of said contact face comprises an adhesive-bearing portion that surrounds a surface of a sebum indicator which changes appearance subsequent to absorbing sebum from skin.

2. The device of claim 1, wherein said sebum indicator makes up between about 5% and about 50% of a surface area of said contact face.

3. The device of claim 1, wherein said sebum indicator comprises a sebum-absorbing hydrophobic microporous film.

4. The device of claim 3, said sebum indicator comprising a single type of said sebum-absorbing hydrophobic microporous film on said contact face.

5. The device of claim 3, said sebum indicator comprising at least two different types of said sebum-absorbing hydrophobic microporous film on said contact face, each different said type of microporous film having different sebum-absorbing properties.

6. The device of claim 3, said sebum indicator comprising a microporous film assembly comprising a vertical spacer having a front side and a back side,
wherein said microporous film is attached to said front side of said vertical spacer; and
wherein said microporous film assembly attached to other components of the device through said back side of said vertical spacer.

7. The device of claim 6, comprising an adhesive layer which constitutes said adhesive-bearing portion of said contact face and which also contacts said back side of said vertical spacer so as to maintain said microporous film on said contact face.

8. The device of claim 6, wherein said vertical spacer has a thickness of between about 50 micrometers and about 250 micrometers.

9. The device of claim 1, said adhesive-bearing portion of said contact face including at least two differently-colored parts when viewed from perpendicular towards said contact face:
a dark-colored part of said contact face constituting a hydration-characterizing portion of said contact face; and
a light-colored part of said contact face constituting a pollution-characterizing portion of said contact face.

10. The device of claim 1, wherein at least one of:
said upper surface of said body being non-glare; and
said contact face being non-glare.

11. The device of claim 1, said upper surface of said body comprising at least one image-correction feature that assists in computerized correction of the color and/or white balance of an acquired image in which said upper surface appears.

12. The device of claim 1, said contact face comprising at least one image-correction feature that assists in computerized correction of a distortion of a portion of an acquired image that corresponds to said contact face.

13. The device of claim 1, said body comprising a supporting sheet which provides said body with at least one property selected from the group consisting of being not-stretchy, flexibility to conform to the irregularly curved surface of human skin, stiffness and elasticity.

14. A method for making a device comprising a sebum indicator according to claim 1, comprising:
a. providing a sheet of material suitable for being a supporting sheet of a body of the device having a front side and a back side;
b. providing a microporous film assembly comprising a vertical spacer having a front side and a back side, with a microporous film attached to said front side of said vertical spacer;
c. covering said front side of said sheet of material with a layer of adhesive;
d. contacting said back side of said vertical spacer of said microporous film assembly with said layer of adhesive covering said front side of said sheet of material where said microporous film assembly is surrounded by a surface of said layer of adhesive, thereby attaching said microporous film assembly to said front side of said sheet of material; and
e. cutting said sheet of material to yield an incipient device comprising a sebum indicator, wherein said cutting is such that a microporous film assembly constituting an incipient sebum indicator of a device is surrounded by a surface of said layer of adhesive constituting an adhesive-bearing portion of a device.

15. The method of claim 14, said sheet of material having a thickness of between about 50 micrometers and about 450 micrometers.

16. The method of claim 14, further comprising prior to ‘c’ applying a colorant to said front side of said sheet of material having a desired color pattern.

17. (canceled)

18. (canceled)

19. (canceled)

20. A device suitable for characterizing skin, comprising:
a body having an upper surface and a lower contact face;
said upper surface and said lower contact face both having an area of not less than 0.2 cm² (equivalent to 0.5 cm diameter circle) and not more than 1020 cm² (equivalent to a 18 cm diameter circle);
said body having a thickness of not less than 30 micrometers and not more than 10 mm, wherein at least a part of said contact face comprises a surface of a sebum indicator which changes appearance subsequent to absorbing sebum from skin,
said sebum indicator comprising at least two different types of said sebum-absorbing hydrophobic microporous film on said contact face, each different said type of microporous film having different sebum-absorbing properties.

21. A device suitable for characterizing skin, comprising:
a body having an upper surface and a lower contact face;
said upper surface and said lower contact face both having an area of not less than 0.2 cm² (equivalent to 0.5 cm diameter circle) and not more than 1020 cm² (equivalent to a 18 cm diameter circle);
said body having a thickness of not less than 30 micrometers and not more than 10 mm, wherein at least a part of said contact face comprises an adhesive-bearing portion that includes at least two differently-colored parts when viewed from perpendicular towards said contact face:
a dark-colored part of said contact face constituting a hydration-characterizing portion of said contact face; and
a light-colored part of said contact face constituting a pollution-characterizing portion of said contact face.
